(19) United States
(12) Patent Application Publication
(10) Pub. No.: US 2024/0158472 A1
(43) Pub. Date: May 16, 2024

(54) FERRITIN VARIANTS WITH INCREASED STABILITY AND COMPLEXATION ABILITY

(71) Applicant: Cellis AG, Switzerland (CH)

(72) Inventors: Alessandro ARCOVITO, Rome (IT); Alessandra BONAMORE, Aprilia (LT) (IT); Alberto BOFFI, Rome (IT); Ilona MARSZALEK, Bramki (PL); Marcin SKORZYNSKI, Zyrardow (PL); Tomasz RYGIEL, Warsaw (PL); Magdalena KROL, Warszawa (PL)

(21) Appl. No.: 18/551,020

(22) PCT Filed: Mar. 18, 2022
(86) PCT No.: PCT/EP2022/057205

§ 371 Date: Sep. 22, 2023

(30) Foreign Application Priority Data
Mar. 18, 2021 (WO) PCT/EP2021/056996

(51) Int. Cl. C07K 14/79; A61K 35/17; A61K 38/00; A61K 47/64

(52) U.S. Cl. CPC C07K 14/79; A61K 35/17; A61K 38/00; A61K 47/644; C07K 2319/60

(57) ABSTRACT

The present invention relates to a ferritin variant polypeptide, wherein at least one lysine residue is deleted or substituted with a non-basic amino acid. The invention further relates to a complex of this polypeptide and a label or drug, and an isolated cellular delivery system comprising the polypeptide or the complex of the invention as well as uses of such system for prophylaxis, therapy, diagnosis or theragnosis, in particular for therapy of cancer or inflammatory diseases.

The present invention relates to new ferritin variants, wherein at least one lysine residue is deleted or substituted with a non-basic amino acid. The invention further relates to a complex of this polypeptide and a compound, a label or drug, and an isolated delivery system comprising the polypeptide or the complex of the invention (alone or as cellular system) as well as uses of such system for prophylaxis, therapy or diagnosis, in particular for therapy of cancer or inflammatory diseases.

### BACKGROUND OF THE INVENTION

Ferritin has emerged as a promising protein-based nanocage thanks to its unique architecture, surface properties and high biocompatibility. However, to date, no successful ferritin drug complexes have reached the clinic (Truffi M et al., Pharmacol Res. 2016 May; 107:57-65). The inventors surprisingly discovered that by deletion or substitution of cysteine residues, several properties of ferritin polypeptides can be significantly improved. One or more problems described in the prior art are solved by the ferritin variant polypeptides defined in the appended claims and in the second aspect of the invention described below. The described mutations (i.e., deletions or substitutions of lysine residues) were found to improve the properties of ferritins as carriers, in particular as nanocages for drugs and/or labels. The inventive ferritin variant polypeptides provide inter alia for: (i) improved encapsulation efficiency of drugs or labels into ferritin nanocages; (ii) improved nucleic acid binding properties of ferritin; (iii) improved stability of ferritin nanocages; (iv) improved stability of drug encapsulation in ferritin nanocages; (v) improved protein recovery after loading of ferritin nanocages; (vi) enhanced cytotoxicity against tumor cells by drugs enclosed within ferritin nanocages or attached to ferritin; (vii) improved chemico-physical properties of ferritin nanocages; (viii) decreased tendency of dimer formation of ferritin nanocages; (ix) decreased tendency of aggregation of ferritin nanocages.

### SUMMARY OF THE INVENTION

In a first aspect the present invention relates to a polypeptide comprising a transferrin receptor binding domain (TRBD) of a ferritin variant wherein within the TRBD the ferritin variant in comparison to the wild-type ferritin on which it is based comprises one or more glutamine residues mutated into glutamic acid residues and/or one or more asparagine residues mutated into aspartic acid residues.

In a second aspect the present invention relates to a ferritin variant polypeptide, wherein at least one, at least two, at least three or at least four, preferably four, lysine residues, preferably lysine residues at position 54, 72, 87 and/or 144 indicated with respect to SEQ ID NO. 1 (human wild-type heavy chain ferritin), are deleted or substituted with a non-basic amino acid.

In a third aspect the present invention relates to a ferritin variant polypeptide, wherein one or more cysteine residues, in particular cysteine residues at position 91, 103 and/or 131 indicated with respect to SEQ ID NO. 1, are deleted or substituted, preferably substituted with serine residues.

In a fourth aspect the present invention relates to a nucleic acid encoding the polypeptide of the first, second or third aspect.

In another aspect the present invention relates to a vector comprising the nucleic acid of the fourth aspect.

In a fifth aspect the present invention relates to a conjugate comprising the polypeptide of the first, second or third aspect and at least one label and/or at least one drug.

In a sixth aspect the present invention relates to a complex comprising at least one polypeptide of the first, second or third aspect and/or at least one conjugate of the fifth aspect of the present invention.

In a seventh aspect the present invention relates to an isolated targeted delivery system comprising a cell, wherein the cell comprises the polypeptide of the first aspect, second or third, the conjugate of the fifth aspect, or the complex of the sixth aspect of the present invention.

In an eighth aspect the present invention relates to a pharmaceutical or diagnostic composition comprising the polypeptide of the first, second or third aspect, the conjugate of the fifth aspect, the complex of the sixth aspect or the isolated targeted delivery system of the seventh aspect and a pharmaceutically acceptable carrier and/or suitable excipient(s).

In a ninth aspect the present invention relates to the polypeptide of the first, second or third aspect, the conjugate of the fifth aspect, the complex of the sixth aspect, the isolated targeted delivery system of the seventh aspect for use in medicine.

In a tenth aspect the present invention relates to the polypeptide of the first, second or third aspect, the conjugate of the fifth aspect, the complex of the sixth aspect, the isolated targeted delivery system of the seventh aspect, or the pharmaceutical or diagnostic composition of the eighth aspect for use in treating, preventing or diagnosing a tumour, preferably a solid tumour and/or its metastases, preferably breast cancer, pancreatic cancer, bladder cancer, lung cancer, colon cancer, ovarian cancer, liver cancer, glioma/glioblastoma or a tumour having hypoxic areas; an inflammatory disease or ischemic areas, in particular in skin wounds or after organ infarctus (heart) or ischemic retina; or for prophylactic or therapeutic vaccination, in particular to prevent or treat an infectious disease or cancer.

In an eleventh aspect the present invention relates to a method of treating, preventing or diagnosing a tumour, preferably a solid tumour and/or its metastases, preferably breast cancer, pancreatic cancer, bladder cancer, lung cancer, colon cancer, ovarian cancer, liver cancer, glioma/glioblastoma or a tumour having hypoxic areas; an inflammatory disease or ischemic areas, in particular in skin wounds or after organ infarctus (heart) or ischemic retina; or a method of prophylactic or therapeutic vaccination, in particular to prevent or treat an infectious disease or cancer by administering an effective amount of the polypeptide of the first, second or third aspect, the conjugate of the fifth aspect, the complex of the sixth aspect or the isolated targeted delivery system of the eighth aspect to a subject in need thereof.

### DETAILED DESCRIPTION OF PREFERRED EMBODIMENTS

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

The terms “peptide” or “polypeptide” are used interchangeably in the context of the present invention to refer to a chain of at least two amino acids linked by peptide bonds. Thus, the term “polypeptide” in the context of the present invention is also used to refer to amino acid chains with more than 50, more than 100 or more than 150 amino acids.

The term “amino acid” encompasses naturally occurring amino acids as well as amino acid derivatives. In the context of the present specification, amino acids are identified either using the 1-letter code (Hausman R E, Cooper G M (2004) or the 3-letter code. The cell: a molecular approach. Washington, D.C.: ASM Press. p. 51. ISBN 978-0-87893-214-6). An amino acid identified with the letter X corresponds to any amino acid. An amino acid identified with the letter B corresponds to either D (asparagine) or N (aspartic acid). An amino acid identified with the letter Z corresponds to either E (glutamine) or Q (glutamic acid).

The terms “polynucleotide” and “nucleic acid” are used interchangeably herein and are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA) and its various forms (e.g. but not limited to ssRNA, LNA etc.), deoxyribonucleic acid (DNA), and mixtures thereof such as e.g. RNA-DNA hybrids. The nucleic acids, can e.g. be synthesized chemically, e.g. in accordance with the phosphotriester method (see, for example, Uhlmann, E. &Peyman, A. (1990) Chemical Reviews, 90, 543-584). “Aptamers” are nucleic acids which bind with high affinity to a polypeptide. Aptamers can be isolated by selection methods such as SELEmir146-a (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability.

The term “identity” is used throughout the specification with regard to polypeptide and nucleotide sequence comparisons. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise. For example, a polypeptide sequence consisting of 200 amino acids compared to a reference 300 amino acid long polypeptide sequence may exhibit a maximum percentage of sequence identity of 66.6% (200/300) while a sequence with a length of 150 amino acids may exhibit a maximum percentage of sequence identity of 50% (150/300). If 15 out of those 150 amino acids are different from the respective amino acids of the 300 amino acid long reference sequence, the level of sequence identity decreases to 45%. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin&Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/ or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). BLAST protein searches are performed with the BLASTP program, score=50, word length=3. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Supp11:I54-I62) or Markov random fields. Structure based alignments for multiple protein sequences and/or structures using information from sequence database searches, available homologs with 3D structures and user-defined constraints may also be used (Pei J, Grishin N V: PROMALS: towards accurate multiple sequence alignments of distantly related proteins. Bioinformatics 2007, 23:802-808; 3DCoffee@igs: a web server for combining sequences and structures into a multiple sequence alignment. Poirot O, Suhre K, Abergel C, O'Toole E, Notredame C. Nucleic Acids Res. 2004 Jul. 1; 32:W37-40.). When percentages of sequence identity are referred to in the present application, these percentages are calculated in relation to the full length of the longer sequence, if not specifically indicated otherwise.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

The term “TRBD” refers to an N-terminal polypeptide fragment of a ferritin polypeptide of between 15 to 40 amino acids, in particular approximately 20 amino acids, that is capable of specifically binding to CD71. Preferably, the TRBD has at least 50% of the binding affinity to CD71 as the full length ferritin polypeptide, preferably at least 75%, more preferably at least 90%. It is well known in the art how to measure the binding affinity between two proteins. Preferably, the affinity between the TRBD and CD71, preferably between TRBD and CD71 of the same species, is measured by surface plasmon resonance at RT. Preferably, the K_{D} of the binding affinity is 100 nM or lower, 50 nM or lower 20 nM or lower or 5 nM or lower.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

In a first aspect the present invention relates to a polypeptide comprising a transferrin receptor binding domain (TRBD) of a ferritin variant wherein within the TRBD the ferritin variant in comparison to the wild-type ferritin on which it is based comprises one or more glutamine residues (E) mutated into glutamic acid residues (Q) and/or one or more asparagine residues (D) mutated into aspartic acid residues (N). The ferritin variant, in particular the TRBD of the ferritin variant, comprises at least the following amino acid sequence:

(SEQ ID NO. 81)
MTTASX₁SZ₁VRZ₂BYHZ₃DX₂EAA

X₁=S or T, preferably T;
X₂=S or A, preferably S;
Z₁, Z₂, and Z₃=Q or E; and
B=N or D;
wherein at least one of Z₂ and Z₃ is E and/or B is D. The amino acid sequence may further comprise one, two or three amino acid substitutions outside Z, in particular outside Z₂ and/or Z₃, and/or B. The M at position 1 may be present or absent. The amino acid sequence according to SEQ ID NO. 81 specifies the TRBD of the ferritin variant. In other words, the polypeptide according to the first aspect of the invention comprises a TRBD according to SEQ ID NO. 81. It is preferred that the polypeptide of the first aspect of the invention is a ferritin polypeptide, i.e. a polypeptide having a similar structure as a wild-type ferritin polypeptide described below and/or an amino acid sequence that is homologous to the sequence of a wild-type ferritin polypeptide described below (e.g. sequence identity of at least 80%, 85%, 90% or 95% to a wild-type ferritin polypeptide described below). The polypeptide according to the first aspect of the invention, i.e. the polypeptide comprising a TRBD of a ferritin variant wherein within the TRBD the ferritin variant in comparison to the wild-type ferritin on which it is based comprises one or more glutamine residues mutated into glutamic acid residues and/or one or more asparagine residues mutated into aspartic acid residues, is also referred to as “TRBD variant ferritin polypeptide” in this specification.

The family of transferrin receptors comprises transferrin receptor 1 (TfR-1) and transferrin receptor 2 (TfR-2). TfR-1 is expressed on all actively proliferating cells, while TfR-2 is expressed mainly on hepatocytes and erythroid precursor cells. Transferrin receptor-I has been shown to mediate cellular uptake of ferritins via endocytosis. The designations “TfR-1”, “CD71”, and “TFRC” are used interchangeably and refer to transferrin receptor-1.

Ferritin is a hollow globular protein complex consisting of 24 ferritin monomer subunits assembled into a cage-like structure. Ferritin is the primary intracellular iron-storage protein. It is produced by almost all living organisms and is present in every cell type. Ferritin genes are highly conserved among species. In vertebrates, two ferritin monomers exist: the light (L) chain and the heavy (H) chain type with a molecular weight of 19 kDa or 21 kDa respectively. Vertebrate ferritin 24-mers can be homo-oligomers consisting of either L or H chains, or hetero-oligomers consisting of both L and H chains (Theil E C, 1987, Annual Review of Biochemistry. 56 (1): 289-315): Typically ferritin complexes have internal and external diameters of about 8 and 12 nm, respectively. Ferritin was shown to be internalized by endocytosis upon binding to CD71. Interaction of ferritin and CD71 is mediated via ferritin-H chains (Li L et al, Proc. Natl. Acad. Sci. USA 107 (8) (2010) 3505-3510). Ferritins are not abundant in plasma, but can be readily produced in high yield as recombinant proteins in common protein expression systems such as Escherichia coli cells.

In the TRBD variant ferritin polypeptides according to the first aspect of the invention, uncharged amino acids (glutamine or asparagine) of the wild-type sequence are substituted with negatively charged amino acids (glutamic acid or aspartic acid). The inventors surprisingly found that this results in an increased affinity to CD71. Without wishing to be bound by any theory, the present inventors consider it likely that the negatively charged mutants establish additional interactions with the transferrin binding part of CD71, thus resulting in an energetically more favourable interaction between CD71 and the TRBD variant ferritin polypeptides.

The TRBD variant ferritin polypeptides according to the first aspect of the invention can be described as “isosteric mutants”, because compared to the respective wildtype polypeptides, they exhibit an identical or very similar geometry. Compared to these “isosteric mutants”, mutants carrying other mutations within the TRBD have a geometry that differs from that of the wildtype. Without wishing to be bound by any theory, the present inventors consider it likely that the resulting lack of surface complementarity with CD71 causes a reduced binding affinity to CD71.

In a preferred embodiment of the polypeptide according to the present invention, the wild-type ferritin is a mammalian ferritin. The mammalian ferritin may be a mouse, rat, dog, ape, in particular chimpanzee, or human ferritin. In a preferred embodiment, the mammalian ferritin is a mouse, rabbit, rat or human ferritin.

In a preferred embodiment of the polypeptide according to the present invention, the wild-type ferritin is a human heavy chain ferritin.

In a preferred embodiment of the polypeptide according to the present invention, the wild-type ferritin has an amino acid sequence selected from the group consisting of SEQ ID NO: 1, which corresponds to the human ferritin heavy chain polypeptide and SEQ ID NO: 2, which corresponds to the murine ferritin heavy chain polypeptide. In a preferred embodiment of the polypeptide according to the present invention, the wild-type ferritin has an amino acid sequence according to SEQ ID NO: 1.

In a preferred embodiment of the polypeptide according to the present invention, the mutations are comprised in the 20 N-terminal amino acids of the wild-type ferritin.

Amino acid substitutions are preferably selected in a way that they do not unduly change the conformation of the polypeptide, as a lack of surface complementarity with TfR-1 will prevent binding of the ferritin variant to CD71. As an example, a “small amino acid” should be substituted with another small amino acid. A “small amino acid” in the context of the present invention is preferably an amino acid having a molecular weight of less than 125 Dalton. Preferably, a small amino acid in the context of the present invention is selected from the group consisting of the amino acids glycine, alanine, serine, cysteine, threonine, and valine, or derivatives thereof. As another example, an amino acid having a hydrophobic side chain should be substituted with another amino acid having a hydrophobic side chain.

In a preferred embodiment of the polypeptide according to the first aspect of the present invention, the ferritin variant, in particular the TRBD of the ferritin variant, comprises at least an amino acid sequence selected from the group consisting of SEQ ID NO. 05 to 18, 20 to 33, 35 to 48 and 50 to 63, which may further comprise one, two or three amino acid substitutions outside amino acid positions 11, 12 and/or 15.

In a preferred embodiment of the polypeptide according to the first aspect of the present invention, the ferritin variant, in particular the TRBD of the ferritin variant, comprises at least an amino acid sequence selected from the group consisting of SEQ ID NO: 05 to SEQ ID NO. 18, which may further comprise one, two or three amino acid substitutions outside amino acid positions 11, 12 and/or 15. SEQ ID NO. 04 to SEQ ID NO. 18 correspond to single, double, triple or quadruple mutants of the human ferritin TRBD sequence.

In a preferred embodiment of the polypeptide according to the first aspect of the present invention, the ferritin variant, in particular the TRBD of the ferritin variant, comprises at least an amino acid sequence selected from the group consisting of SEQ ID NO. 05, 11, 12, 15, 20, 26, 27, 30, 35, 41, 42, 45, 50, 56, 57 and 60, which may further comprise one, two or three amino acid substitutions outside amino acid positions 11, 12 and/or 15.

In a preferred embodiment of the polypeptide according to the first aspect of the present invention, the ferritin variant, in particular the TRBD of the ferritin variant, comprises at least an amino acid sequence selected from the group consisting of SEQ ID NO. 05, 11, 20, 26, 35, 41, 50 and 56, which may further comprise one, two or three amino acid substitutions outside amino acid position 11. These sequences correspond to mutants Q11E and 2ECSE (Q11E Q15E).

In a preferred embodiment of the polypeptide according to the first aspect of the present invention, the ferritin variant, in particular the TRBD of the ferritin variant, comprises at least an amino acid sequence selected from the group consisting of SEQ ID NO. 05, 20, 35 and 50, which may further comprise one, two or three amino acid substitutions outside amino acid position 11. SEQ ID NO. 05, 20, 35 and 50 correspond to mutant Q11E.

In a preferred embodiment of the polypeptide according to the first aspect of the present invention, the ferritin variant, in particular the TRBD of the ferritin variant, comprises at least an amino acid sequence selected from the group consisting of SEQ ID NO. 11, 26, 41 and 56, which may further comprise one, two or three amino acid substitutions outside amino acid positions 11 and 12. SEQ ID NO. 11, 26, 41 and 56 correspond to mutant EDCSE (Q11E N12D).

In a preferred embodiment of the polypeptide according to the first aspect of the present invention, the ferritin variant, in particular the TRBD of the ferritin variant, comprises at least an amino acid sequence selected from the group consisting of SEQ ID NO. 12, 27, 42 and 57, which may further comprise one, two or three amino acid substitutions outside amino acid positions 11 and 15. SEQ ID NO. 12, 27, 42 and 57 correspond to mutant 2ECSE (Q11E Q15E).

In a preferred embodiment of the polypeptide according to the first aspect of the present invention, the ferritin variant, in particular the TRBD of the ferritin variant, comprises at least an amino acid sequence selected from the group consisting of SEQ ID NO. 15, 30, 45 and 60, which may further comprise one, two or three amino acid substitutions outside amino acid positions 8, 11 and 15. SEQ ID NO. 15, 30, 45 and 60 correspond to mutant 3ECSE (Q8E Q11E Q15E).

In a preferred embodiment of the polypeptide according to the first aspect of the present invention, the ferritin variant, in particular the TRBD of the ferritin variant, comprises at least SEQ ID NO. 05 (mutant Q11E), which may further comprise one, two or three amino acid substitutions outside amino acid position 11.

In a preferred embodiment of the polypeptide according to the first aspect of the present invention, the ferritin variant, in particular the TRBD of the ferritin variant, comprises at SEQ ID NO. 11, which may further comprise one, two or three amino acid substitutions outside amino acid positions 11 and 12.

In a preferred embodiment of the polypeptide according to the first aspect of the present invention, the ferritin variant, in particular the TRBD of the ferritin variant, comprises at least SEQ ID NO. 12, which may further comprise one, two or three amino acid substitutions outside amino acid positions 11 and 15.

In a preferred embodiment of the polypeptide according to the first aspect of the present invention, the ferritin variant, in particular the TRBD of the ferritin variant, comprises at least SEQ ID NO. 15, which may further comprise one, two or three amino acid substitutions outside amino acid positions 8, 11 and 15.

In a preferred embodiment of the polypeptide according to the present invention, in addition to the TRBD, the polypeptide further comprises an amino acid sequence having at least 90%, 95%, 97%, 98%, 99% or 100%, such as 90%, 95%, 97%, 98%, 99% or 100%, identity to a sequence selected from the group consisting of SEQ ID NO. 64 to SEQ ID NO. 70, SEQ ID NO. 78 to SEQ ID NO. 80 and SEQ ID NO. 87. Preferably, this amino acid sequence is comprised C-terminally of the TRBD.

The polypeptide of the present invention essentially retains the properties of a wild-type ferritin polypeptide with regard to complex formation (cage-like structure consisting of 24 ferritin monomer subunits) and uptake of iron. The expression “essentially retains” is meant to include embodiments in which complex/24mer formations is improved compared to the wild-type.

SEQ ID NO. 64 is an N-terminally truncated consensus sequence based on sequences of mammalian H-type ferritins. Within SEQ ID NO. 64 X at position 1 may be present or absent, if present it means any amino acid, preferably I, X at position 2 means any amino acid, preferably N, X at position 9 means any amino acid, preferably Y, X at position 19 means any amino acid, preferably C or Y, more preferable Y, X at position 61 means any amino acid, preferably F, X at position 63 means any amino acid, preferably Q, X at position 70 means any amino acid, preferably R or C, more preferably C, X at position 85 means any amino acid, preferably H, X at position 89 means any amino acid, preferably S or N, more preferably N, X at position 116 may be present or absent, if present it means any amino acid, preferably Y or H, more preferably H, X at position 119 means any amino acid, preferably S or N, more preferably N, X at position 124 means any amino acid, preferably S or A, more preferably A, X at position 143 means any amino acid, preferably A or S, more preferably A, X at position 145 means any amino acid, preferably M or L, more preferably L, X at position 157 means any amino acid, preferably H or D, more preferably D, X at position 160 may be absent or any amino acid, preferably N, X at position 161 may be any amino acid, preferably E, X at position 162 may be any amino acid, preferably S.

SEQ ID NO. 65 is an N-terminally and C-terminally truncated consensus sequence based on sequences of mammalian H-type ferritins. Within SEQ ID NO. 65 X at position 1 may be present or absent, if present it means any amino acid, preferably I, X at position 2 means any amino acid, preferably N, X at position 9 means any amino acid, preferably Y, X at position 19 means any amino acid, preferably C or Y, more preferable Y, X at position 61 means any amino acid, preferably F, X at position 63 means any amino acid, preferably Q, X at position 70 means any amino acid, preferably R or C, more preferably C, X at position 85 means any amino acid, preferably H, X at position 89 means any amino acid, preferably S or N, more preferably N, X at position 116 may be present or absent, if present it means any amino acid, preferably Y or H, more preferably H, X at position 119 means any amino acid, preferably S or N, more preferably N, X at position 124 means any amino acid, preferably S or A, more preferably A.

SEQ ID NO. 66 is an alternative N-terminally truncated consensus sequence based on sequences of mammalian H-type ferritins. Within SEQ ID NO. 66 X at position 1 means any amino acid, preferably N, X at position 8 means any amino acid, preferably Y, X at position 60 means any amino acid, preferably F, X at position 62 means any amino acid, preferably Q, X at position 84 means any amino acid, preferably H, X at position 123 means any amino acid, preferably S or A, more preferably A, X at position 159 may be absent or any amino acid, preferably N, X at position 160 may be any amino acid, preferably E, X at position 161 may be any amino acid, preferably S.

In a preferred embodiment of the polypeptide according to the present invention, the polypeptide comprises an amino acid sequence having at least 90%, 95%, 97%, 98%, 99% or 100%, such as 90%, 95%, 97%, 98%, 99% or 100%, identity to a sequence selected from the group consisting of SEQ ID NO. 67 to SEQ ID NO. 70.

In a preferred embodiment of the polypeptide according to the present invention, the polypeptide comprises an amino acid sequence having at least 90%, 95%, 97%, 98%, 99% or 100% identity to a sequence comprising amino acids 2-118 of SEQ ID NO. 67, SEQ ID NO. 68, SEQ ID NO. 69 or SEQ ID NO. 70.

In a preferred embodiment of the polypeptide according to the present invention, the polypeptide comprises an amino acid sequence having at least 90%, 95%, 97%, 98%, 99% or 100% identity to SEQ ID NO. 67. Within SEQ ID NO. 67, I at position 1 may be present or absent.

In a preferred embodiment of the polypeptide according to the present invention, the polypeptide comprises an amino acid sequence having at least 90%, 95%, 97%, 98%, 99% or 100% identity to a sequence comprising amino acids 2-118 of SEQ ID NO. 67.

In a preferred embodiment of the polypeptide according to the present invention, the polypeptide comprises, essentially consists of or consists of a sequence selected from the group consisting of SEQ ID NO. 05 to SEQ ID NO. 63, which may further comprise one, two or three amino acid substitutions outside amino acid positions 11, 12 and/or 15; and a sequence having at least 90%, 95%, 97%, 98%, 99% or 100% identity to a sequence selected from the group consisting of SEQ ID NO. 64 to SEQ ID NO. 70, SEQ ID NO. 78 to SEQ ID NO. 80 and SEQ ID NO. 87 or a sequence having 90%, 95%, 97%, 98%, 99% or 100% identity to a sequence consisting of amino acids 1-118 or 2-118 of SEQ ID NO. 64 to SEQ ID NO. 70, SEQ ID NO. 78 to SEQ ID NO. 80, or SEQ ID NO. 87, in particular SEQ ID NO. 67, SEQ ID NO. 68, SEQ ID NO. 69, SEQ ID NO. 70, SEQ ID NO. 78 to SEQ ID NO. 80, or SEQ ID NO. 87, more particularly SEQ ID NO. 78 to SEQ ID NO. 80, or SEQ ID NO. 87, most particularly SEQ ID NO. 80.

In a preferred embodiment of the polypeptide according to the present invention, the polypeptide comprises, essentially consists of or consists of a sequence selected from the group consisting of SEQ ID NO: 05 to SEQ ID NO. 18 which may further comprise one, two or three amino acid substitutions outside amino acid positions 8, 11, 12 and/or 15 and a sequence having at least 90%, 95%, 97%, 98%, 99% or 100% identity to SEQ ID NO. 67.

It is preferred that the polypeptide of the first aspect of the invention comprises further mutations compared to a wild-type ferritin sequence in the amino acid sequences outside the TRBD. In preferred embodiments, one, two, three or four, preferably four, lysine residues, preferably lysine residues at position 54, 72, 87 and/or 144 indicated with respect to SEQ ID NO. 1 (human wild-type heavy chain ferritin), are deleted or substituted with a non-basic amino acid. A substitution with a non-basic amino acid is preferred over a deletion. A non-basic amino acid may be an acidic amino acid, such as D or E, an uncharged polar amino acid, such as S, T, N or Q, or an uncharged non-polar amino acid. Preferred is a substitution with E or Q. Most preferably, K54 is substituted with E, K72 is substituted with E, K87 is substituted with Q and K144 is substituted with E. The inventors surprisingly discovered that these mutations enhance the purification efficiency, decrease contamination, in particular contamination with nucleic acids, more particularly DNA, and endotoxins, increase the stability and improve the encapsulation efficiency (examples 3-8).

In some embodiments of the polypeptide of the first aspect of the invention, one, two or three, preferably two or three, cysteine residues, preferably cysteine residues at position 91, 103 and/or 131 indicated with respect to SEQ ID NO. 1 (human wild-type heavy chain ferritin), are deleted or substituted, preferably substituted with serine residues. The inventors surprisingly discovered that these mutations decrease the aggregation of ferritin polypeptides into high molecular weight complexes and improves the formation of 24-mers (examples 3-4). In some embodiments of the polypeptide of the first aspect of the invention one or more lysine residues at position 54, 72, 87 and/or 144 and one or more cysteine residues at position 91, 103 and/or 131 indicated with respect to SEQ ID NO. 1 are deleted or substituted as described above.

In other embodiments, the cysteine residues are not mutated or deleted, as they can be used for covalent conjugation to drugs or labels, as claimed in the fifth aspect of the invention.

In a preferred embodiment of the polypeptide according to the present invention, the polypeptide comprises, essentially consists of or consists of an amino acid sequence selected from the group consisting of SEQ ID NO. 71, SEQ ID NO. 72, SEQ ID NO. 73, SEQ ID NO. 74 SEQ ID NO. 75, SEQ ID NO. 76, and SEQ ID NO. 77 or an amino acid sequence having at least 90%, 95%, 97%, 98%, or 99% identity to one of SEQ ID NO. 71-77. The polypeptide has at least the same affinity to TfR-1 and/or at least the same ability to form 24mers as wild-type human heavy chain ferritin according to SEQ ID NO. 2.

In a preferred embodiment of the polypeptide according to the present invention, the polypeptide comprises, essentially consists of or consists of SEQ ID NO. 71. In a preferred embodiment of the polypeptide according to the present invention, the polypeptide comprises, essentially consists of or consists of SEQ ID NO. 72. In a preferred embodiment of the polypeptide according to the present invention, the polypeptide comprises, essentially consists of or consists of SEQ ID NO. 73. In a preferred embodiment of the polypeptide according to the present invention, the polypeptide comprises, essentially consists of or consists of SEQ ID NO. 74.

In an even more preferred embodiment of the polypeptide according to the present invention, the polypeptide comprises, essentially consists of or consists of SEQ ID NO. 75, SEQ ID NO. 76, or SEQ ID NO.77, most preferably SEQ ID NO. 77, or an amino acid sequence having at least 90%, 95%, 97%, 98%, or 99% identity to one of SEQ ID NO. 75-77, preferably to SEQ ID NO. 77.

In a preferred embodiment of the polypeptide according to the present invention, the affinity of the TRBD to TfR-1 is increased in comparison to the TRBD of the wild-type ferritin to TfR-1 at least (≥) 1.5×, ≥2×, ≥3×, ≥4×, ≥5×, ≥10×, ≥20×, ≥30×, ≥40×, ≥50×, but less than (≤) 60×, ≤50×, ≤40×, ≤30×, ≤20×, ≤10×, or ≤5×. In this and the following embodiments, “TRBD of the wild-type ferritin” refers to the TRBD, in particular amino acids 1-20, of the human or murine ferritin heavy chain polypeptide according to SEQ ID NO. 1 or 2, respectively. In a preferred embodiment, the affinity of the TRBD to TfR-1 is increased at least 1.5× in comparison to the affinity of the TRBD of the wild-type ferritin to TR. In a preferred embodiment, the affinity of the TRBD to TfR-1 is increased at least 2× in comparison to the affinity of the TRBD of the wild-type ferritin to TfR-1. In a preferred embodiment, the affinity of the TRBD to TfR-1 is increased at least 3× in comparison to the affinity of the TRBD of the wild-type ferritin to TfR-1. In a preferred embodiment, the affinity of the TRBD to TfR-1 is increased at least 4× in comparison to the affinity of the TRBD of the wild-type ferritin to TfR-1. In a preferred embodiment, the affinity of the TRBD to TfR-1 is increased at least 5× in comparison to the affinity of the TRBD of the wild-type ferritin to TfR-1. In a preferred embodiment, the affinity of the TRBD to TfR-1 is increased at least 10× in comparison to the affinity of the TRBD of the wild-type ferritin to TfR-1. In a preferred embodiment, the affinity of the TRBD to TfR-1 is increased at least 20× in comparison to the affinity of the TRBD of the wild-type ferritin to TfR-1. In a preferred embodiment, the affinity of the TRBD to TfR-1 is increased at least 30× in comparison to the affinity of the TRBD of the wild-type ferritin to TfR-1. In a preferred embodiment, the affinity of the TRBD to TfR-1 is increased at least 40× in comparison to the affinity of the TRBD of the wild-type ferritin to TfR-1. In a preferred embodiment, the affinity of the TRBD to TfR-1 is increased at least 50× in comparison to the affinity of the TRBD of the wild-type ferritin to TfR-1. In a preferred embodiment, the affinity of the TRBD to TfR-1 is increased less than 60× in comparison to the affinity of the TRBD of the wild-type ferritin to TfR-1. In a preferred embodiment, the affinity of the TRBD to TfR-1 is increased less than 50× in comparison to the affinity of the TRBD of the wild-type ferritin to TfR-1. In a preferred embodiment, the affinity of the TRBD to TfR-1 is increased less than 40× in comparison to the affinity of the TRBD of the wild-type ferritin to TfR-1. In a preferred embodiment, the affinity of the TRBD to TfR-1 is increased less than 30× in comparison to the affinity of the TRBD of the wild-type ferritin to TfR-1. In a preferred embodiment, the affinity of the TRBD to TfR-1 is increased less than 20× in comparison to the affinity of the TRBD of the wild-type ferritin to TfR-1. In a preferred embodiment, the affinity of the TRBD to TfR-1 is increased less than 10× in comparison to the affinity of the TRBD of the wild-type ferritin to TfR-1. In a preferred embodiment, the affinity of the TRBD to TfR-1 is increased less than 5× in comparison to the affinity of the TRBD of the wild-type ferritin to TfR-1. In a preferred embodiment, the affinity of the TRBD to TfR-1 is increased between 1.5×-50× in comparison to the TRBD of the wild-type ferritin. In a preferred embodiment, the affinity of the TRBD to TfR-1 is increased between 2×-50× in comparison to the TRBD of the wild-type ferritin. In a preferred embodiment, the affinity of the TRBD to TfR-1 is increased between 3×-50× in comparison to the TRBD of the wild-type ferritin. In a preferred embodiment, the affinity of the TRBD to TfR-1 is increased between 4×-50× in comparison to the TRBD of the wild-type ferritin. In a preferred embodiment, the affinity of the TRBD to TfR-1 is increased between 5×-50× in comparison to the TRBD of the wild-type ferritin. In a preferred embodiment, the affinity of the TRBD to TfR-1 is increased between 10×-50× in comparison to the TRBD of the wild-type ferritin. In a preferred embodiment, the affinity of the TRBD to TfR-1 is increased between 20×-50× in comparison to the TRBD of the wild-type ferritin. In a preferred embodiment, the affinity of the TRBD to TfR-1 is increased between 30×-50× in comparison to the TRBD of the wild-type ferritin. In a preferred embodiment, the affinity of the TRBD to TfR-1 is increased between 40×-50× in comparison to the TRBD of the wild-type ferritin. In a preferred embodiment, the affinity of the TRBD to TfR-1 is increased between 1.5×-10× in comparison to the TRBD of the wild-type ferritin. In a preferred embodiment, the affinity of the TRBD to TfR-1 is increased between 2×-20× in comparison to the TRBD of the wild-type ferritin. In a preferred embodiment, the affinity of the TRBD to TfR-1 is increased between 5×-30× in comparison to the TRBD of the wild-type ferritin.

An increased binding affinity of the TRBD to TfR-1 is advantageous because it favors binding of the TRBD variant ferritin polypeptides to TfR-1. This increases the amount of TRBD variant ferritin polypeptides bound to TfR-1 expressed on the surface of a cell within a given time and/or a given concentration of ferritin. If an active ingredient is conjugated to TRBD variant ferritin polypeptides or encapsulated within oligomers of TRBD variant ferritin polypeptides, increased binding of TRBD variant ferritin polypeptides to TfR-1 facilitates loading of a cell expressing TfR-1 with the active ingredient.

In order to exert its function, an active ingredient conjugated to a TRBD variant ferritin polypeptide or encapsulated within an oligomer of TRBD variant ferritin polypeptides has to be released eventually. Without wishing to be bound by theory, the acidic pH of the (late) endosomal compartment may lead to disassembly of ferritin oligomers and thus to release of the active ingredient encapsulated within the oligomers. The present inventors have also found that the binding affinity of TRBD to TfR-1 should not be increased excessively, in order not to completely prevent dissociation of the TRBD variant ferritin polypeptide from TfR-1. In order to be optimal, the increase in affinity should not reach two orders of magnitude.

In a second aspect the present invention relates to a ferritin variant polypeptide, wherein at least one, at least two, at least three or at least four, preferably four, lysine residues, preferably lysine residues at position 54, 72, 87 and/or 144 indicated with respect to SEQ ID NO. 1 (human wild-type heavy chain ferritin), are deleted or substituted with a non-basic amino acid. A substitution with a non-basic amino acid is preferred over a deletion. A non-basic amino acid may be an acidic amino acid, such as D or E, an uncharged polar amino acid, such as S, T, N or Q, or an uncharged non-polar amino acid. The ferritin variant polypeptide is characterized by a structural and functional homology to a wild-type ferritin as defined above. The inventors surprisingly discovered that these mutations enhance the purification efficiency, decrease contamination, in particular contamination with nucleic acids, more particularly DNA, and endotoxins, increase the stability and improve the encapsulation efficiency (examples 3-8). In some embodiments, the ferritin variant polypeptide of the second aspect comprises a mutation (i.e. a deletion or substitution) at position 54 and 72, or 54 and 87, or 54 and 144, or 72 and 87, or 72 and 144, or 87 and 144, or 54, 72 and 87, or 54, 72 and 144, or 54, 87 and 144, or 72, 87 and 144, or 54, 72, 87 and 144. It is preferred that the ferritin variant polypeptide comprises a mutation at position 54, 72, 87 and/or 144. Most preferably, K54 is substituted with E, K72 is substituted with E, K87 is substituted with Q and K144 is substituted with E.

It is preferred that the ferritin variant polypeptide of the second aspect of the invention has a sequence according to SEQ ID NO. 82 (mammalian consensus), SEQ ID NO. 1 (human heavy chain ferritin) or SEQ ID NO. 2 (murine heavy chain ferritin), wherein at least one, preferably all, lysine residues at position 54, 72, 87 and/or 144 are deleted or substituted with a non-basic amino acid, preferably E or Q, preferably the K at position 54 is substituted with E, the K at position 72 is substituted with E, the K at position 87 is substituted with Q and/or the K at position 144 is substituted with E. The sequences according to SEQ ID NO. 82, SEQ ID NO. 1 and SEQ ID NO. 2 may further comprise 1-5, e.g. 1, 2, 3, 4 or 5, 1-10, e.g. 1, 2, 3, 4, 5, 6, 7, 8, 9 or 10, 1-15, 1-20 or 1-25 amino acid mutations outside position 54, 72, 87 and/or 144.

In SEQ ID NO. 82, which is a mammalian consensus sequence, X at position 6 can be any naturally occurring amino acid, preferably Pro, X at position 14 can be any naturally occurring amino acid, preferably His, X at position 16 can be any naturally occurring amino acid, preferably Asp, X at position 21 may be present or absent, if present it means any amino acid, preferably Ile, X at position 22 means any amino acid, preferably Asn, X at position 30 can be any naturally occurring amino acid, preferably Tyr, X at position 40 can be any naturally occurring amino acid, preferably Tyr or Cys, more preferably Tyr, X at position 82 can be any naturally occurring amino acid, preferably Phe, X at position 84 can be any naturally occurring amino acid, preferably Gln, X at position 91 can be any naturally occurring amino acid, preferably Arg or Cys, more preferably Cys, X at position 106 can be any naturally occurring amino acid, preferably His, X at position 110 can be any naturally occurring amino acid, preferably Asn or Ser, more preferably Asn, X at position 137 can be any naturally occurring amino acid, preferably His or Tyr, more preferably His, X at position 140 can be any naturally occurring amino acid, preferably Asn or Ser, more preferably Asn, X at position 145 can be any naturally occurring amino acid, preferably Ala or Ser, more preferably Ala, X at position 164 can be any naturally occurring amino acid, preferably Ala or Ser, more preferably Ser, X at position 166 can be any naturally occurring amino acid, preferably Met or Leu, preferably Leu, X at position 178 can be any naturally occurring amino acid, preferably Asp or His, more preferably Asp, X at position 181 is absent or any naturally occurring amino acid, preferably Asn, X at position 182 is absent or any naturally occurring amino acid, preferably Glu, X at position 183 is absent or any naturally occurring amino acid, preferably Ser.

Preferably, the ferritin variant polypeptide of the second aspect of the invention has a sequence according to SEQ ID NO. 82, SEQ ID NO. 1 or SEQ ID NO. 2 comprising substitution K54E or K72E or K87Q or K144E, or K54E and K72E, or K54E and K87Q, or K54E and K144E, or K72E and K87Q, or K72E and K144E, or K87Q and K144E, or K54E, K72E and K87Q, or K54E, K87Q and K144E, or K54E, K72E and K144E, or K72E, K87Q and K144E, or K54E, K72E, K87Q and K144E, preferably K54E, K72E, K87Q and K144E, wherein the sequences according to SEQ ID NO. 82, SEQ ID NO. 1 and SEQ ID NO. 2 may further comprise 1-5, e.g. 1, 2, 3, 4 or 5, 1-10, e.g. 1, 2, 3, 4, 5, 6, 7, 8, 9 or 10, 1-15, 1-20 or 1-25 amino acid mutations outside position 54, 72, 87 and/or 144.

In preferred embodiments, the ferritin variant polypeptide of the second aspect of the invention further comprises isosteric mutations in the TRBD, in particular mutation Q8E, Q11E, N12D and/or Q15E, preferably Q11E or Q11E and Q15E. The isosteric mutations are indicated with respect to the human wild-type ferritin sequence according to SEQ ID NO. 1 and are described in the first aspect of the invention.

In some embodiments, in the ferritin variant polypeptide of the second aspect of the invention, one or more cysteine residues, in particular cysteine residues at position 91, 103 and/or 131, are deleted or mutated, preferably mutated to serine residues.

In most preferred embodiments, the ferritin variant polypeptide of the second aspect of the invention has a sequence according to SEQ ID NO. 83, SEQ ID NO. 84, SEQ ID NO. 85, SEQ ID NO. 86, SEQ ID NO. 75, SEQ ID NO. 76, or SEQ ID NO. 77 or a sequence according to SEQ ID NO. 83, SEQ ID NO. 84, SEQ ID NO. 85, SEQ ID NO. 86, SEQ ID NO. 75, SEQ ID NO. 76, or SEQ ID NO. 77 comprising 1-5, e.g. 1, 2, 3, 4 or 5, or 1-10, e.g. 1, 2, 3, 4, 5, 6, 7, 8, 9 or 10 amino acid mutations outside position 54, 72, 87 and/or 144. In particularly preferred embodiments, the ferritin variant polypeptide of the second aspect of the invention has a sequence according to SEQ ID NO. 77.

SEQ ID NO. 86 is a mammalian consensus sequence, wherein each X has the same meaning as indicated above for SEQ ID NO: 82.

In a third aspect the present invention relates to a ferritin variant polypeptide, wherein one or more cysteine residues, in particular cysteine residues at position 91, 103 and/or 131 indicated with respect to SEQ ID NO. 1, are deleted or substituted, preferably substituted with serine residues. The ferritin variant polypeptide is characterized by a structural and functional homology to a wild-type ferritin as defined above. The inventors surprisingly discovered that these mutations decrease the aggregation of ferritin polypeptides into high molecular weight complexes and improves the formation of 24mers (examples 3-4).

It is preferred that the ferritin variant polypeptide of the third aspect of the invention has a sequence according to SEQ ID NO. 82, SEQ ID NO. 1 or SEQ ID NO. 2, wherein at least one, preferably all, cysteine residues at position 91, 103 and/or 131 are mutated, preferably mutated to serine residues. The sequences according to SEQ ID NO. 82, SEQ ID NO. 1 and SEQ ID NO. 2 may further comprise 1-5, e.g. 1, 2, 3, 4 or 5, 1-10, e.g. 1, 2, 3, 4, 5, 6, 7, 8, 9 or 10, 1-15, 1-20 or 1-25 amino acid mutations outside position 91, 103 and/or 131.

In SEQ ID NO. 82, which is a mammalian consensus sequence, the X comprised in the sequence have the meaning outlined above.

In preferred embodiments, the ferritin variant polypeptide of the third aspect of the invention further comprises isosteric mutations in the TRBD, in particular mutation Q8E, Q11E, N12D and/or Q15E, preferably Q11E or Q11E and Q15E. The isosteric mutations are indicated with respect to the human wild-type ferritin sequence according to SEQ ID NO. 1 and are described in the first aspect of the invention.

In preferred embodiments, the ferritin variant polypeptide of the third aspect of the invention further comprises a mutation at position 54, 72, 87 and/or 144. Preferably, the mutations are substitutions, in particular a substitution to E at position 54, a substitution to E at position 72, a substitution to Q at position 87 and/or a substitution to E at position 144. These mutations are further described in the second aspect of the invention.

In most preferred embodiments, the ferritin variant polypeptide of the third aspect of the invention has a sequence according to SEQ ID NO. 75 or SEQ ID NO. 76 or a sequence according to SEQ ID NO. 75 or SEQ ID NO. 76 comprising 1-5, e.g. 1, 2, 3, 4 or 5, or 1-10, e.g. 1, 2, 3, 4, 5, 6, 7, 8, 9 or 10 amino acid mutations outside position 91, 103 and/or 131.

Active ingredients may be conjugated to the TRBD variant ferritin polypeptides of the first aspect of the invention or the polypeptides of the second or third aspect of the invention or may be encapsulated in oligomers of the TRBD variant ferritin polypeptides of the first aspect of the invention or the polypeptides of the second or third aspect of the invention. The term “active ingredient” encompasses therapeutically active ingredients and/or diagnostically active ingredients. Thus, the term “active ingredient” refers to a therapeutic agent (also referred to as drug) and/or to a diagnostic agent (also referred to as label). The inventors noted that the TRBD variant ferritin polypeptides according to the invention represent preferred constructs to specifically deliver active ingredients, in particular encapsulated active ingredients, to cells expressing TfR-1. Furthermore, the inventors noted that ferritin variants according to the invention are able to deliver active ingredients, in particular encapsulated active ingredients, to the cell nucleus.

The polypeptide according to the present invention may comprise additional domains. In a preferred embodiment of the polypeptide according to the present invention, the polypeptide further comprises an antigen binding domain, in particular an antibody or antibody fragment.

The term “antibody” as used in the context of the present invention refers to a glycoprotein belonging to the immunoglobulin superfamily; the terms antibody and immunoglobulin are often used interchangeably. An antibody refers to a protein molecule produced by plasma cells and is used by the immune system to identify and neutralize foreign objects such as bacteria and viruses. The antibody recognizes a unique part of the foreign target, its antigen.

The term “antibody fragment” as used herein, refers to one or more fragments of an antibody that retain the ability to specifically bind to an antigen. Examples of binding fragments encompassed within the term “antibody fragment” include a fragment antigen binding (Fab) fragment, a Fab′ fragment, a F(ab′)₂ fragment, a heavy chain antibody, a single-domain antibody (sdAb), a single-chain fragment variable (scFv), a fragment variable (Fv), a V_{H} domain, a V_{L}domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, an alternative scaffold protein, and a fusion protein thereof.

The term “diabody” as used within this specification refers to a fusion protein or a bivalent antibody, which can bind different antigens. A diabody is composed of two single protein chains, which comprise fragments of an antibody, namely variable fragments. Diabodies comprise a heavy chain variable domain (V_{H}) connected to a light-chain variable domain (V_{L}) on the same polypeptide chain (V_{H}-V_{L}, or V_{L}-V_{H}). By using a short peptide connecting the two variable domains, the domains are forced to pair with the complementary domain of another chain and thus, create two antigen-binding sites. Diabodies can target the same (monospecific) or different antigens (bispecific).

A “single domain antibody”, refers to antibody fragments consisting of a single, monomeric variable domain of an antibody. Simply, they only comprise the monomeric heavy chain variable regions of heavy chain antibodies produced by camelids or cartilaginous fish. Due to their different origins they are also referred to VHH or VNAR (variable new antigen receptor-fragments. Alternatively, single-domain antibodies can be obtained by monomerization of variable domains of conventional mouse or human antibodies by the use of genetic engineering. They show a molecular mass of approximately 12-15 kDa and thus, are the smallest antibody fragments capable of antigen recognition. Further examples include nanobodies or nanoantibodies.

The term “antibody mimetic” as used within the context of the present specification refers to compounds, which can specifically bind antigens, similar to an antibody, but are not structurally related to antibodies. Usually, antibody mimetics are artificial peptides or proteins with a molar mass of about 3 to 20 kDa, which comprise one, two or more exposed domains specifically binding to an antigen. Examples include inter alia the LACI-D1 (lipoprotein-associated coagulation inhibitor); affilins, e.g. human-γ B crystalline or human ubiquitin; cystatin; Sac7D from Sulfolobusacidocaldarius; lipocalin and anticalins derived from lipocalins; DARPins (designed ankyrin repeat domains); SH3 domain of Fyn; Kunitz domain of protease inhibitors; monobodies, e.g. the 10^{th} type III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric ortrimeric human C-type lectin domain; microbodies, e.g. trypsin-inhibitor-II; affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

The term “antigen” is used to refer to a substance, preferably an immunogenic peptide that comprises at least one epitope, preferably an epitope that elicits a B or T cell response or B cell and T cell response.

An “epitope”, also known as antigenic determinant, is that part of a substance, e.g. an immunogenic polypeptide, which is recognized by the immune system. Preferably, this recognition is mediated by the binding of antibodies, B cells, or T cells to the epitope in question. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. The term “epitope” comprises both conformational and non-conformational epitopes. Conformational and non-conformational epitopes are distinguished in that the binding to the former but not the latter is lost in the presence of denaturing solvents.

An immunogenic polypeptide according to the present invention is, preferably, derived from a pathogen selected from the group consisting of viruses, bacteria and protozoa. However, in an alternative embodiment of the present invention the immunogenic polypeptide is a tumour antigen, i.e. polypeptide or fragment of a polypeptide specifically expressed by a cancer.

In a fourth aspect the present invention relates to a nucleic acid encoding the polypeptide of the first, second or third aspect.

In another aspect the present invention relates to a vector comprising the nucleic acid of the fourth aspect.

In a fifth aspect the present invention relates to a conjugate comprising the polypeptide of the first, second or third aspect of the present invention and at least one label and/or at least one drug.

The phrase “conjugate comprising polypeptide and at least one label and/or at least one drug” as used in the context of the present invention refers to a composition in which one or more molecules of the active ingredient are covalently or non-covalently bound to a polypeptide of the first, second or third aspect of the invention. The covalent or non-covalent binding between the polypeptide and the active ingredient can be direct or indirect. In the latter case the active ingredient is linked to the polypeptide via a linker or spacer. Linker or spacers are known to the skilled artisan, such as polyalanine, polyglycine, carbohydrates, (CH₂)ₙ groups or polypeptide linkers, in particular peptide based cleavable linkers (e.g. cathepsin sensitive valine-citrulline sequence and para-aminobenzylcarbamate spacer). The skilled artisan will thus be able to select the respective suitable linker(s) or spacer(s) depending on the respective application.

In a sixth aspect the present invention relates to a complex comprising at least one polypeptide of the first, second or third aspect of the present invention and/or at least one conjugate of the fifth aspect of the present invention.

The phrase “complex comprising polypeptide and/or at least one conjugate” as used in the context of the present invention refers to a complex formed by one or more polypeptides of the first, second or third aspect of the invention, by one or more conjugates of the fifth aspect of the invention, or by at least one polypeptide of the first, second or third aspect of the invention and at least one conjugate of the fifth aspect of the invention. The complex is formed by covalent or non-covalent binding between the polypeptide(s) and/or the conjugate(s). The covalent or non-covalent binding can be direct or indirect. In a preferred embodiment, the complex is an oligomer, in particular a 24-mer, formed by non-covalent binding between the polypeptide(s) and/or the conjugate(s).

In a preferred embodiment of the complex according to the invention, the complex further comprises at least one label and/or at least one drug.

The terms “drug” or “therapeutic agent” are used synonymously in the context of the present invention and refer to any compound that modifies or modulates cell activity or is capable of being activated, i.e. a prodrug, to modify or modulate cell activity, preferably in the body of a patient. Examples of such active ingredients include so called “small molecules” and peptides. The term “small molecule” is used in the context of the present invention to refer to a hydrocarbon with a molecular mass of below 1.500 g/mol or to pharmaceutically active radioactive isotopes. Preferred, drugs that can be used comprise anti-cancer drugs, pharmaceutically active radioactive isotopes or ferrihydrite.

The term “prodrug” as used in the context of the present invention refers to any active ingredient that, after administration, is metabolized or otherwise converted to a biologically active or more active ingredient (or drug) with respect to at least one property. In comparison to the drug, a prodrug is modified chemically in a manner that makes it, relative to the drug, less active or inactive, but the chemical modification is such that the corresponding drug is generated by metabolic or other biological processes after the prodrug is administered to the patient. A prodrug may for example have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

The terms “label” or “diagnostic agent” are used interchangeably herein and refer to any kind of compound being suitable for diagnostic purposes. Preferred compounds are selected from a fluorescent dye, a radioisotope and a contrast agent. A contrast agent is a dye or other substance that helps to show abnormal areas inside the body. In one embodiment the term label refers to a compound that comprises a chelating agent which forms a complex with divalent or trivalent metal cations. Preferred radioisotopes/fluorescence emitting isotopes are selected from the group consisting of alpha radiation emitting isotopes, gamma radiation emitting isotopes, Auger electron emitting isotopes, X-ray emitting isotopes, fluorescent isotopes, such as ⁶⁵Tb, fluorescence emitting isotopes, such as ¹⁸F, ⁵¹Cr, ⁶⁷Ga, ⁶⁸Ga, ¹¹¹In, ^{99m}Tc, ¹⁴⁰La, ⁷⁵Yb, ¹⁵³Sm, ¹⁶⁶Ho, ⁸⁸Y, ⁸⁹Zr, ⁹⁰Y, ¹⁴⁹Pm, ¹⁷⁷Lu, ⁴⁷Sc, ¹⁴²Pr, ¹⁵⁹Gd, ²¹²Bi, ⁷²As, ⁷²Se, ⁹⁷Ru, ¹⁰⁹Pd, ¹⁰⁵Rh, ^{101m15}Rh, ¹¹⁹Sb, ¹²⁸Ba, ¹²³I, ¹²⁴I, ¹³¹I, ¹⁹⁷Hg, ²¹¹At, ¹⁶⁹Eu, ²⁰³Pb, ²¹²Pb, ⁶⁴Cu, ⁶⁷Cu, ¹⁸⁸Re, ¹⁸⁶Re, ¹⁹⁸Au and ¹⁹⁹Ag as well as conjugates and combinations of above with proteins, peptides, small molecular inhibitors, antibodies or other compounds, e.g. ¹⁸Ffluorodeoxyglucose (¹⁸F-FDG) or ⁶⁴Cu-porfirin. Preferred fluorescent dyes are selected from the following classes of dyes: Xanthens (e.g. Fluorescein), Acridines (e.g. Acridine Yellow), Oxazines (e.g. Oxazine 1), Cynines (e.g. Cy7/Cy 3), Styryl dyes (e.g. Dye-28), Coumarines (e.g. Alexa Fluor 350), Porphines (e.g. Chlorophyll B), Metal-Ligand-Complexes (e.g. PtOEPK), Fluorescent proteins (e.g. APC, R-Phycoerythrin), Nanocrystals (e.g. QuantumDot 705), Perylenes (e.g. Lumogen Red F300) and Phtalocyanines (e.g. IRDYE™700DX) as well as conjugates and combinations of these classes of dyes or fluorescent ⁶⁵Tb emitting. Preferred contrast agents are selected from paramagnetic agents, e.g. Gd, Eu, W and Mn, preferably complexed with a chelating agent. Further options are superparamagnetic iron (Fe) complexes and particles, compounds containing atoms of high atomic number, i.e. iodine for computer tomography (CT), microbubbles and carriers such as liposomes that contain these contrast agents.

The at least one label and/or at least one drug (i.e. at least one active ingredient) is covalently or non-covalently bound to a TRBD variant ferritin polypeptide according to the first aspect of the invention or a polypeptide according to the second or third aspect of the invention or is encapsulated within the complex according to the sixth aspect of the invention. Thus, the term “complex” also encompasses the enclosure of active ingredients within the cage even in the absence of a covalent or non-covalent bond between the protein(s) and the active ingredient(s). The formation of the complex allows the transport of the active ingredients into the cell when the cell is internalizing the ferritin. Thus, it is preferred that the active ingredients are bound to the iron binding protein in a way that does not interfere with the transport mechanism. This can be easily tested by the skilled person using uptake assays known in the art and described in the Example Section below. If the complex comprising an active ingredient is taken up by a cell and transported to a target region within the body, it is preferred that the complex is sufficiently stable to survive the transport within the cell to the target region within the body. Thus, it is preferred that the complex rather than the active ingredient alone is delivered to the cells or into the cells in the target region. This property also reduces possible deleterious effects, e.g. cytotoxicity, of the active ingredient to the cell delivering the active ingredient.

Active ingredients can be encapsulated within the internal cavity of a ferritin oligomer (physical confinement) by exploiting the association/dissociation properties of the ferritin macromolecule itself. The active ingredients are held in place by non-covalent interactions with amino acid residues within the cavity internal surface.

If active ingredients are covalently coupled to TRBD variant ferritin polypeptides or polypeptides according to the second or third aspect of the invention such coupling is preferably through amino acids residues known to be located in surface areas that are not involved in binding of ferritin to TfR-1. TRBD variant ferritin polypeptides used in the context of the present invention can form stable non-covalently bound complexes with a wide variety of active ingredients. If the active ingredient is a peptide, e.g. an antigenic peptide, it is preferred that it is not expressed as a fusion with the iron binding protein, since in this case release of the peptide from the iron binding protein will require endosomal processing of the entire ferritin peptide fusion protein.

Whatever the conjugation/adsorption/binding method, the TRBD variant ferritin polypeptides or polypeptides according to the second or third aspect of the invention and the described conjugates and complexes thereof were shown by the present inventors to be privileged carriers of drugs and labels, once loaded into appropriate cell systems with tumour targeting properties, e.g. activated macrophages. The purification procedure of these TRBD variant ferritin polypeptides or polypeptides according to the second or third aspect of the invention is easy, fast, cheap and safe, which provides a tremendous added value.

The following preferred embodiments further specify both the fifth and sixth aspect of the present invention.

In a preferred embodiment, the drug and/or label is selected from the group consisting of a protein, a nucleic acid, a chemical non-protein non-nucleic acid compound with a molecular weight of less than 1.5 kDa, more preferably less than 1 kDa, a virus, and a α- or β-radiation emitting radioisotope, which also emits a cell damaging amount of γ-radiation.

If the drug is a nucleic acid it is preferred that it is a miRNA, siRNA, chemically modified-RNA, LNA, ssRNA, DNAzyme or a nucleic acid encoding a pharmaceutically active protein, e.g. an antibody, an antibody mimetic, a cytokine, a prodrug-converting enzyme, an immunogenic peptide or the like.

In a preferred embodiment, the label is selected from the group consisting of a fluorescent dye, a radioisotope/fluorescence emitting isotope, a detectable polypeptide or nucleic acid encoding a detectable polypeptide, and a contrast agent.

In a preferred embodiment, the fluorescent dye is selected from the group consisting of the following classes of fluorescent dyes: Xanthens, Acridines, Oxazines, Cynines, Styryl dyes, Coumarines, Porphines, Metal-Ligand-Complexes, Fluorescent proteins, Nanocrystals, Perylenes and Phtalocyanines as well as conjugates and combinations of these classes of dyes.

In a preferred embodiment, the radioisotope/fluorescence emitting isotope is selected from the group consisting of alpha radiation emitting isotopes, gamma radiation emitting isotopes, Auger electron emitting isotopes, X-ray emitting isotopes, fluorescent isotopes, such as 65Tb, fluorescence emitting isotopes, such as 18F, 51Cr, 67Ga, 68Ga, 89Zr, 111In, 99mTc, 140La, 175Yb, 153Sm, 166Ho, 88Y, 90Y, 149Pm, 177Lu, 47Sc, 142Pr, 159Gd, 212Bi, 72As, 72Se, 97Ru, 109Pd, 105Rh, 101m15Rh, 119Sb, 128Ba, 123I, 124I, 131I, 197Hg, 211At, 169Eu, 203Pb, 212Pb, 64Cu, 67Cu, 188Re, 186Re, 198Au and 199Ag as well as conjugates and combinations of above with proteins, peptides, small molecular inhibitors, antibodies or other compounds (e.g. ¹⁸F-FDG, ⁸⁹Zr-oxide or ⁶⁴Cu-porfirin).

In a preferred embodiment, the detectable polypeptide is an autofluorescent protein, preferably green fluorescent protein or any structural variant thereof with an altered adsorption and/or emission spectrum.

In a preferred embodiment, the contrast agent comprises a paramagnetic agent, preferably selected from Gd, Eu, W and Mn, or ferrihydride.

In a preferred embodiment, the label comprises a chelating agent which forms a complex with divalent or trivalent metal cations.

In a preferred embodiment of the targeted delivery system of the present invention the chelating agent is selected from the group consisting of 1,4,7,10-tetraazacyclododecane-N,N′,N,N′-tetraacetic acid (DOTA), ethylenediaminetetraacetic acid (EDTA), 1,4,7-triazacyclononane-1,4,7-triacetic acid (NOTA), triethylenetetramine (TETA), iminodiacetic acid, Diethylenetriamine-N,N,N′,N′,N″-pentaacetic acid (DTPA) and 6-Hydrazinopyridine-3-carboxylic acid (HYNIC).

In a preferred embodiment, the drug is selected from the group consisting of an anticancer drug, an anti arteriosclerotic drug, and an anti-inflammatory drug or immunomodulatory drug (e.g. TRL agonists, STING agonists, mimicking viral or bacterial infection).

In a preferred embodiment, the anticancer drug is a cytostatic drug, cytotoxic drug or prodrug thereof.

Preferred anticancer drugs are selected from an apoptosis/autophagy or necrosis-inducing drug. An apoptosis/autophagy or necrosis-inducing drug can be any drug that is able to induce apoptosis/autophagy or necrosis effectively even in cells having an abnormality in cell proliferation. These drugs are preferably used in complexes with one or more ferritins.

In a preferred embodiment, the anticancer drug is selected from the group consisting of an apoptosis-inducing drug, an alkylating substance, anti-metabolites, antibiotics, epothilones, nuclear receptor agonists and antagonists, an anti-androgene, an anti-estrogen, a platinum compound, a hormone, a antihormone, an interferon, an inhibitor of cell cycle-dependent protein kinases (CDKs), an inhibitor of cyclooxygenases and/or lipoxygenases, a biogeneic fatty acid, a biogenic fatty acid derivative, including prostanoids and leukotrienes, an inhibitor of protein kinases, an inhibitor of protein phosphatases, an inhibitor of lipid kinases, a platinum coordination complex, an ethyleneimine, a methylmelamine, a triazine, a vinca alkaloid, a pyrimidine analog, a purine analog, an alkylsulfonate, a folic acid analog, an anthracendione, a substituted urea, and a methylhydrazin derivative, an ene-diyne antibiotic, a tubulin polymerization inhibitor such as a maytansinoid or an auristatine derivate, immune check-point inhibitor, and an inhibitor of tumour-specific protein or marker, preferably a Rho-GDP-dissociation inhibitor, more preferably Grp94, or AXL inhibitor.

In a preferred embodiment, the anticancer drug is selected from the group consisting of acediasulfone, aclarubicine, ambazone, aminoglutethimide, auristatin, L-asparaginase, azathioprine, banoxantrone, bendamustine, bleomycin, busulfan, calcium folinate, carboplatin, carpecitabine, carmustine, celecoxib, chlorambucil, cis-platin, cladribine, cyclophosphamide, cytarabine, dacarbazine, dactinomycindapsone, daunorubicin, dibrompropamidine, diethylstilbestrole, docetaxel, doxorubicin, enediynes, epirubicin, epothilone B, epothilone D, estramucin phosphate, estrogen, ethinylestradiole, etoposide, flavopiridol, floxuridine, fludarabine, fluorouracil, fluoxymesterone, flutamidefosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon α, irinotecan, leuprolide, lomustine, lurtotecan, mafenidesulfateolamide, mechlorethamine, medroxyprogesterone acetate, megastrolacetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, scriflavinium chloride, semustinestreptozocine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, tinidazole, topotecan, triaziquone, treosulfan, trimethoprim, trofosfamide, UCN-01, vinblastine, vincristine, vindesine, vinblastine, vinorelbine, and zorubicin, preferably selected from the group consisting of auristatin, banoxantrone, bendamustine, chlorambucil, chaliceamycin, cyclophosphamidedynemycin A, maytansine, melphalan, mertansine, and neocazinostatin, most preferably banoxantrone, bendamustine, chlorambucil, cyclophosphamide, pyrrolobenzodiazepine and melphalan.

In a preferred embodiment, the anticancer drug is a proliferation inhibiting protein, preferably a cell cycle inhibitor or an antibody or antibody like binding protein that specifically binds to a proliferation promoting protein or a nucleic acid, preferably encoding a proliferation inhibiting protein or an antibody or antibody like binding protein that specifically binds to a proliferation promoting protein or a siRNA or DNAzyme.

In a preferred embodiment, the immunomodulatory drug activates or inhibits the activity of immune cells. These can be natural or synthetic ligands, including antibodies, or antagonists of Pattern Recognition Receptors, particularly Toll-like Receptors, NOD-like receptors (NLR), RIG-I-like receptors (RLR). Physiologically, these receptors recognize class of signals known as pathogen-associated molecular patterns (PAMPs) and damage-associated molecular patterns (DAMPs).

Preferred examples of antibodies to be used in the context of the present invention are single chain antibodies, antibody fragments, nanobodies, light or heavy chains, variable light or variable heavy chains, or diabodies. Preferred antibody fragments comprise a fragment antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, and a fusion protein thereof.

In a preferred embodiment, the virus is an oncolytic virus.

In a preferred embodiment, the α or β radiation emitting radioisotope, which also emits a cell damaging amount of γ radiation is selected from the group consisting of lutetium-177, ytterbium-90, iodine-131, samarium-153, phosphorus-32, caesium-131, palladium-103, radium-233, iodine-125, and boron-10 or a cell damaging amount of α radiation, preferably selected from the group consisting of actinium-225, bismuth-213, lead-212, and polonium-212. Also preferred is a complex of above mentioned compounds and isotopes linked to the nanoparticles (e.g. gold, argentum, graphen) or these nanoparticles.

In a preferred embodiment, the drug is a hypoxia-activated prodrug, preferably selected from the group consisting of benzotriazine N-oxides, apaziquone (E09), tirapazamine (TPN), SN30000, PR-104A, TH-302, TH-4000, AQ4N.

In a preferred embodiment, the drug is an antigen or a nucleic acid encoding an antigen.

In a preferred embodiment, the bond(s) between the TRBD variant ferritin polypeptide or polypeptides according to the second or third aspect of the invention and the active ingredient in the conjugate are covalent and/or non-covalent; and/or the active ingredient comprised in the complex is entrapped/encapsulated by the oligomers of the TRBD variant ferritin polypeptide. In one embodiment the covalent and/or non-covalent coupling is indirect through a linker or spacer. If the formation of covalent bonds is desired, relevant thiol, amino or carboxyl groups of the TRBD variant ferritin polypeptides are used to covalently couple active ingredients modified by specific active linker moieties reactive towards thiol or amino groups directly or indirectly to the TRBD variant ferritin polypeptides.

TRBD variant ferritin polypeptides or polypeptides according to the second or third aspect of the invention may be linked to cysteine thiol reactive active ingredients bearing a peptide based cleavable linker (e.g. cathepsin sensitive valine-citrulline sequence and para-aminobenzylcarbamate spacer). As a notable example, the antimitotic agent monomethylauristatin E (MMAE) has been used. The peptide-based linker binds the protein to the cytotoxic compound in a stable manner so the drug is not easily released from the protein under physiologic conditions and help prevent toxicity to healthy cells and ensure dosage efficiency. The ferritin active ingredient adduct thus generated is capable of attaching to the selected receptor type, i.e. TfR-1 for ferritin. Once bound the ferritin active ingredient adduct is internalised by endocytosis and thus selectively taken up by targeted cells. The vesicle containing the drug is fused with lysosomes and lysosomal cysteine proteases, particularly cathepsin B start to break down the valine-citrulline linker and MMAE is no longer bound to ferritin and is released directly into the tumour environment.

Alternatively, DM1-SMCC is an efficient mertansine derivative bearing a linker that specifically binds to lysine residues generating a covalent complex with ferritin, in a reaction that has been successfully described for antibodies. In particular, ferritin can be reacted with DM1-SMCC thus providing a covalent protein-drug adduct that can be cleaved inside cells and releases the active drug in a time-dependent manner. The suppression of microtubule dynamics by DM1 induces mitotic arrest and cell death.

The term “full load” is used in the context of the present invention to refer to the maximum amount of ferritin complexed with an active ingredient that can be taken up by the cell of the active delivery system.

It is also envisioned that different pharmaceutically active substances, labels or pharmaceutically active substances and labels are comprised in the complex according to the third aspect of the invention. For example, one type of active ingredient may be bound to a TRBD variant ferritin polypeptide or polypeptides according to the second or third aspect of the invention (non-covalently bound), while another type is encapsulated in the complex. This approach utilizes different release rates of the active ingredients from the complex once delivered to the targeted tissue and/or cells. For example, an active ingredient can be covalently attached to a ferritin molecule either on the surface of the 24-mer or within the internal cavity by exploiting the reactivity of relevant thiol, amino or carboxyl groups. The types of such useful reactions are well known in the art and can be adopted by the person skilled in the art to the particular active ingredient without any additional work. Examples of such reactions are described in Behrens C R, Liu B. Methods for site-specific drug conjugation to antibodies. MAbs. 2014 January-February; 6(1):46-53.

In theragnostic applications, i.e. in which the complex comprises both a label and a drug, it is preferred that the label is covalently attached to the iron binding protein and the drug is non-covalently bound to the iron binding protein and/or entrapped in the internal cavity formed upon assembly of the multimer of TRBD variant ferritin polypeptides or polypeptides according to the second or third aspect of the invention.

In a sixth aspect, the present invention relates to an isolated targeted delivery system comprising a cell, wherein the cell comprises the polypeptide of the first, second or third aspect, the conjugate of the fifth aspect or the complex of the sixth aspect of the present invention.

The term “targeted delivery system” refers to a system that is capable of delivering an active ingredient to the targeted region, i.e. of capable of targeted delivery, preferably within the body of a patient.

The term “targeted delivery” refers to the delivery of a therapeutic or diagnostic agent (herein together referred to also as “active ingredient”) to a subject, e.g. patient, in particular to a cell within the body of a patient. Targeted delivery also includes “targeted theragnostic delivery”, meaning that both a therapeutic and a diagnostic agent are delivered concomitantly, preferably to a diseased region, thus allowing simultaneous treatment and diagnosis and/or treatment monitoring.

Targeted delivery results in an increased concentration of the active ingredient in a particular region of the body when compared to other regions of the body of that patient. Preferably, the relative concentrations are compared between a diseased region(s) of the body and other regions of the body having similar access to the blood circulation. In preferred embodiments the concentration of the active ingredient in a given number of cells or a given biopsy volume from the diseased region is at least 10% higher, if compared to the identical number of cells or biopsy volume from a non-diseased region after administration of the targeted delivery system of the present invention, preferably after 2-24 hrs. More preferably, the concentration of the active ingredient in the diseased region of the body of a patient is at least 20%, at least 30%, at least 40%, at least 50%, at least 60%, at least 70% at least 80%, at least 90%, at least 100%, at least 150%, at least 200%, at least 250%, at least 300%, at least 350%, at least 400%, at least 450%, at least 500%, more preferably at least 1000% higher than in a non-diseased region of the body after administration of the targeted delivery system of the present invention, preferably after 2-24 hrs. When assessed on the basis of total body distribution it is preferred that at least 5% of the active ingredient administered to a patient is delivered to the diseased region of the body, preferably at least 10%, more preferably at least 15%. The targeted delivery of the active ingredient limits the potential deleterious effects of an active ingredient to the diseased region of the body.

The targeted delivery system according to the present invention enables tumour delivery of the pharmaceutically active substances, labels or pharmaceutically active substances and labels, which normally would not be able to reach the tumour (for example, due to solubility problems). This allows precise administration of the active ingredients to the tumour site (especially to the hypoxic regions) and into the tumour mass, avoiding their accumulation in other organs.

Targeted delivery encompasses both direct and indirect targeting. Direct targeting refers to direct uptake of an active ingredient (as a conjugate according to the second aspect of the invention or in a complex according to the third aspect of the invention) by a diseased cell, e.g. a cancer cell. Indirect targeting refers to delivery of an active ingredient (as a conjugate according to the second aspect of the invention or in a complex according to the third aspect of the invention) to a diseased cell, e.g. a cancer cell, by another cell, e.g. a leukocyte cell.

Mutant Q11E was shown to be capable of at least fourfold higher binding affinity to the CD71 receptor and a corresponding slow rate of release from its complex. The mutant is thus readily taken up by cancer cells overexpressing CD71 receptors (direct targeting). In addition, the mutant is readily taken up by CD45+ leukocytes capable of transferring the ferritin mutants to target cells (indirect targeting).

In particular if the cells of the targeted delivery system of the present invention are leukocytes, the targeted delivery system targets lymph nodes, which makes it particularly suitable for delivery of antigens to dendritic cells residing in the lymph nodes. The lymph node targeting is particularly pronounced, if the cells loaded with the complex are macrophages in particular activated macrophages, even more preferably CCL-2 activated bone marrow derived activated macrophages, or lymphocytes, in particular B cells or T cells. Thus in a preferred embodiment the targeted delivery system is used to deliver one or more antigens in order to elicit a prophylactic and/or therapeutic immune response against the one or more antigens. Preferred antigens are derived from pathogens, i.e. bacteria or viruses or are tumour specific antigens. The term “tumour specific antigens” refers to proteins or epitopes (including peptides with altered glycosylation patterns) that are higher expressed on tumour cells in comparison to non-tumour cells, preferably to antigens or epitopes only expressed on tumour cells. Preferred antigens are selected from the group consisting of epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family; discoidin domain receptor (DDR) family; RET receptor family; KLG receptor family; RYK receptor family; MuSK receptor family; Transforming growth factor α (TGF-α) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), β-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: β-Dgalactose 2-α-Lfucosyltransferase (LDLR/FUT) fusion protein, HLA-A2. arginine to isoleucine exchange at residue 170 of the α-helix of the α2-domain in the HLA-A2 gene (HLA-A*201-R170I), HLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class I, NFYC, OGT, OS-9, pml-RARalpha fusion protein, PRDX5, PTPRK, K-ras (KRAS2), N-ras (NRAS), HRAS, RBAF600, SIRT2, SNRPD1, SYT-SSX1 or -SSX2 fusion protein, triosephosphate isomerase, BAGE, BAGE-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-1, LAGE, LAGE-1, CTL-recognized antigen on melanoma (CAMEL), MAGE-A1 (MAGE-1), MAGE-A2, MAGE-A3, MAGE-A4, MAGE-A5, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pmel17 (SILV), tyrosinase (TYR), TRP-1, HAGE, NA-88, NY-ESO-1, NY-ESO-1/LAGE-2, SAGE, Sp17, SSX-1,2,3,4, TRP2-INT2, carcino-embryonic antigen (CEA), kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBB1), HER-2/neu (ERBB2), interleukin 13 receptor α2 chain (IL13Ralpha2), IL-6 receptor, intestinal carboxyl esterase (iCE), alpha-feto protein (AFP), M-CSF, mdm-2, MUC1, p53 (TP53), PBF, PRAME, PSMA, RAGE-1, RNF43, RU2AS, SOX10, STEAP1, survivin (BIRC5), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA661, LDHC, MORC, SGY-1, SPO11, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), 3-human chorionic gonadotropin, 1-2 microglobulin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins-LMP1, LMP2; others), Hepatitis B or C virus proteins, and HIV proteins.

The targeted delivery system of the present invention has particular suitability to deliver active ingredients to hypoxic areas. Hypoxia is characteristic of various disease including cancer and inflammatory diseases and thus allows targeting such diseases.

In addition to the targeting the use of active ingredients, which are activated under hypoxic conditions adds a further specificity to the targeting and/or further reduces adverse effects of the active ingredients. Thus, in particularly preferred embodiments the active ingredient is a hypoxia-activated prodrug. The backbone of all the hypoxia-activated prodrugs is the presence of one of five different chemical moieties (nitro groups, quinines, aromatic and aliphatic N-oxides and transition metals) that are enzymatically reduced under hypoxic conditions in tissue. Hypoxia-activated prodrugs are any prodrug that is less active or inactive, relative to the corresponding drug, and comprises the drug and one or more bioreducible groups. Such hypoxia-activated prodrugs include all prodrugs activated by a variety of reducing agents and reducing enzymes, including without limitation single electron transferring enzymes (such as cytochrome P450 reductases) and two electron transferring (or hydride transferring) enzymes. According to preferred embodiment of the invention hypoxia-activated prodrug is TH-302. Methods of synthesizing TH-302 are described in PCT application WO 07/002931 and WO 08/083101. Preferably examples of such prodrugs are selected from the class I group consisting of benzotriazine N-oxides, apaziquone (E09), tirapazamine (TPN) and SN30000; or class II group consisting of: nitro compounds PR-104A, TH-302, TH-4000, and AQ4N.

A striking observation was the disease specific homing of the targeted delivery system of the present invention. In particular the CD45⁺ leukocyte cells appear to have a tropism for hypoxic areas and areas of oxidative stress. Hypoxia is a hallmark of various diseases as is oxidative stress. Accordingly, the present invention also relates to an isolated targeted delivery system of the fourth aspect of the invention for use in preventing, treating or diagnosing a disease characterized by hypoxic areas within the diseased tissue and/or by areas of oxidative stress, in particular hypoxic tumours or a hypoxic area within a tumour, or any area within an organism subjected to hypoxic conditions, for example during ischaemic incidents, or undergoing an inflammatory process. Similarly, the invention relates to a method of treating, preventing or diagnosing a disease characterized by hypoxic areas within the diseased tissue and/or by areas of oxidative stress, in particular hypoxic tumours or a hypoxic area within a tumour, or any area within an organism subjected to hypoxic conditions, for example during ischaemic incidents, or undergoing an inflammatory process, by administering an effective amount of the isolated targeted delivery system of the fourth aspect of the invention to a subject in need thereof.

The ability of a given cell or of a population thereof to internalize ferritin depends on the expression of receptors involved in this internalization process. Receptors that lead to internalization of ferritin comprise, e.g. TfR, CXCR4, scavenger receptors, CD163, and TIM-2. The skilled person is well aware how to measure the amount of ferritin uptake and preferred methods of measuring the uptake are described in the Example Section below.

In a preferred embodiment of the isolated targeted delivery system according to the invention, the cell is a CD45⁺ leukocyte, in particular a CD45⁺ leukocyte selected from the group consisting of a monocyte, a differentiated monocyte, a monocyte-macrophage, a lymphocyte and a granulocyte.

The term “leukocyte” (or “leukocyte cell”) is used in the context of the present invention to refer to cells of the immune system that are involved in protecting the body against both infectious disease and foreign invaders. All leukocytes are produced and derived from multipotent cells in the bone marrow known as a hematopoietic stem cells. Leukocytes are found throughout the body, including the blood and lymphatic system. All leukocytes have nuclei, which distinguishes them from the other blood cells, the anucleated red blood cells (RBCs) and platelets. Types of leukocyte can be classified in standard ways. Two pairs of the broadest categories classify them either by structure (granulocytes or agranulocytes) or by cell division lineage (myeloid cells or lymphoid cells). These broadest categories can be further divided into the five main types: neutrophils, eosinophils, basophils, lymphocytes, and monocytes. These types are distinguished by their physical and functional characteristics. Monocytes and neutrophils are phagocytic. Further subtypes can be classified; for example, among lymphocytes, there are B cells, T cells, and NK cells. Granulocytes are distinguished from agranulocytes by their nucleus shape (lobed versus round, that is, polymorphonuclear versus mononuclear) and by their cytoplasm granules (present or absent, or more precisely, visible on light microscopy or not thus visible). The other dichotomy is by lineage: Myeloid cells (neutrophils, monocytes, eosinophils and basophils) are distinguished from lymphoid cells (lymphocytes) by hematopoietic lineage (cellular differentiation lineage).

CD45⁺ expression is characteristic of a subgroup of leukocyte cells, i.e. monocyte, monocyte-macrophages, lymphocytes, granulocytes, NK cells that are suitable to be used in the context of the targeted delivery system of the present invention, in particular since CD45⁺ leukocyte cells are attracted to particular tissues and cells within the body and are capable of delivering complexes of one or more iron binding proteins and one or more pharmaceutically active substances, labels or pharmaceutically active substances and labels to or into cells. This subgroup of leukocytes is in the following referred to as “CD45⁺ leukocyte cells” or “CD45⁺ leukocytes”. Preferably the monocyte is not a dendritic cell which differentiation is controlled by one or more of the following transcription factors: IFN-regulatory factor 8 (IRF8), nuclear factor interleukin (IL)-3-regulated protein (NFIL3), basic leucine zipper transcriptional factor ATF-like 3 (BATF3) or Transcription Factor RelB (NF-KB Subunit)—RELB, Spi-1 Proto-Oncogene (PU/1), recombining binding protein suppressor of hairless (RBPJ), IFN-regulatory factor 4 (IRF4) or transcription factor E2-2 (also known as (TCF4).

It is understood by the skilled person that CD45⁺ leukocyte cells as defined above unless of clonal origin are a mixed population of different leukocytes which share the common property of expressing CD45⁺ surface antigen. Accordingly, subpopulations of cells within the diverse group of CD45⁺ leukocyte cells as defined above are characterized throughout the specification by further functional and/or structural characteristics. The term “CD45⁺” indicates that the majority of cells within a population of cells or essentially all cells express the CD45⁺ surface antigen.

“Expressing” means in this respect that the majority of cells within a population of cells or essentially all cells express the marker (also called surface antigen herein). In this context and also with reference to other cellular surface antigens, the term “expresses” indicates that the surface antigen is produced within the cell and detectably exposed on the surface of a cell. The level of expression and, thus the number of surface antigens detectably exposed on the surface of a cell can vary greatly among different cells. Generally, a cell is considered to be positive, i.e. is indicated to be “⁺”, for a cellular surface antigen, if at least 5, preferably at least 10 copies of the surface antigen are detectably exposed on the surface of the cell. The skilled person is well aware of how to detect, quantify and select for cells, which are positive (or negative) for a given cellular surface antigen. Preferred methods include Fluorescence Activated Cell Sorting (FACS). In this technology fluorescently labelled antibodies are used to bind to cellular surface antigens of a population of cells, the cells are subsequently isolated into single cells and based on fluorescence intensity measured for the single cell, characterized as being positive or negative for the given cellular surface antigen. In some embodiments of the present invention it is indicated that the expression of a given protein is high or low. This means that the protein is detectably expressed in both instances, i.e. is “⁺”, however, at different levels. High and low expression, respectively, will mean different absolute numbers of proteins per cell for different proteins. Thus, a given protein may be considered to be expressed at high levels if there are more than 500 detectable copies of that protein per cell and to be expressed at low levels if there are between 1 to 50 detectable copies of that protein per cell. However, another protein may be considered to be expressed at high levels, if there are more than 5000 detectable copies and expressed at low levels, if there are between 1 to 500 detectable copies per cell. It is well known in the art how to quantify the number of proteins expressed or produced in a cell using flow cytometry and Becton Dickinson Quantibrite™ bead method (see e.g. Pannu, K. K., 2001, Cytometry. 2001 Dec. 1; 45(4):250-8) or mass spectrometry (see, e.g. Milo, R., 2013, Bioessays, 35(12): 1050-1055).

For the purpose of the present invention the term “high expression” of a given protein refers to detectable expression of that protein that is at least 70% of the highest expression level found, i.e. number of copies per cell, in a population of healthy cells, in particular CD45⁺ leukocytes. The term “low expression” of a given protein refers to detectable expression of that protein that is 30% or less of the highest expression level found, i.e. number of copies of that protein per cell, in a population of healthy cells, in particular CD45⁺ leukocytes. Preferably, the “highest expression level” is determined as the average of the highest expression levels found in healthy cells, in particular CD45⁺ leukocytes of different subjects. In some embodiments preferred subpopulations of cells are characterized as “producing” a given protein. This is understood to mean that the protein is not necessarily detectable on the surface of the cell but may only be present inside the cell. The skilled person is well aware how to detect and/or quantify production of a protein inside a cell and/or select cells producing such proteins. Alternatively, cell populations can be defined by expression of specific transcription factors. It is well known in the art how to determine expression of a given protein or its encoding mRNA in a population of cells or even in single cells, e.g. using in vivo labelling with antibodies, FISH assays, in vivo single molecule fluorescent microscopy (Crawford, R. et al. Biophys J. (2013) 105(11): 2439) alone or in combination with Fluorescent Activated Cell Sorting (FACS), or by the PrimeFlow technique (e Bioscience), (Adam S. Venable, et. al., (2015) Methods in Molecular Biology).

The term “differentiated monocyte” is used in the context of the present invention to refer to a monocyte differentiated from the committed precursor termed macrophage-DC precursor (MDP) mainly resident in bone marrow (but could be also in the spleen) and differentiate into either dendritic cells or macrophages. In mice they consist of two main subpopulations: (i) CD11b⁺ cell with high expression of CX3CR1, low expression of CCR2 and Ly6C⁻ and (ii) CD11b⁺ cell with low expression of CX3CR1, high expression of CCR2 and Ly6C⁺. After leaving the bone marrow, mouse Ly6C⁺ monocytes differentiate into Ly6C⁻ monocytes in circulation. Similarly, in human monocyte differentiation, it is accepted that CD14⁺⁺ classical monocytes leave bone marrow and differentiate into CD14⁺⁺ CD16⁺ intermediate monocytes and sequentially to CD14⁺ CD16⁺⁺ non-classical monocytes in peripheral blood circulation (Yang et al. 2014; Biomark Res 2(1) doi. 10.1186/2050-7771-2-1). Preferably the differentiated monocyte is not a dendritic cell, which differentiation is controlled by one or more of the following transcription factors: IRF8, NFIL3, BATF3, RELB, PU/1, RBPJ, IIRF4, and/or TCF4, and more preferably is not a dendritic cell.

Macrophages are tissue-resident professional phagocytes and antigen-presenting cells (APC), which differentiate from circulating peripheral blood monocytes (PBMs). The term “activated macrophage” is used in the context of the present invention to refer to any macrophage that is polarized. Macrophage activation is in general achieved by incubation with interleukins, cytokines and/or growth factors. In particular IL-4 and M-CSF can be used as activating agents. Activated macrophages of different phenotypes are classified into M1-macrophages, classically activated macrophages (CAM) and M2-macrophages, alternatively activated macrophages (AAM). The classically activated M1-macrophages comprise immune effector cells with an acute inflammatory phenotype. These are highly aggressive against bacteria and produce large amounts of lymphokines (Murray, and Wynn, 2011, J LeukocBiol, 89(4):557-63). The alternatively activated, anti-inflammatory M2-macrophages can be separated into at least three subgroups. These subtypes have various different functions, including regulation of immunity, maintenance of tolerance and tissue repair/wound healing. The term “M1 inducer” is used in the context of the present invention to refer to a compound that directs differentiation of PBMs to macrophages of the M1 type. The term “M2 inducer” is used in the context of the present invention to refer to a compound that directs differentiation of PBMs to macrophages of the M2 type. The skilled person is aware of a large number of ways to promote differentiation into either M1 or M2 macrophages.

The term “phagocytosis by macrophages” is the process by which a macrophage engulfs a solid particle to form an internal vesicle known as a phagosome.

Preferably the CD45⁺ monocyte is not a dendritic cell, which differentiation is controlled by one or more of the following transcription factors: IRF8, NFIL3, BATF3, RELB, PU/1, RBPJ, IIRF4, and/or TCF4, and more preferably is not a dendritic cell.

In a preferred embodiment of the isolated targeted delivery system, the CD45⁺ leukocyte cell is producible from a CD34+ hematopoietic precursor cell.

In a preferred embodiment of the of the isolated targeted delivery system
(i) the monocyte is a CD11b⁺ monocyte, preferably selected from the group consisting of a CD11b⁺ CD14⁺ monocyte, a CD11b⁺ CD16⁺ monocyte, a CD11b⁺ CD14⁺ CD16⁺ monocyte, a CD11b⁺ CD14⁺ MHCII⁺ monocyte, a CD11b⁺ CD14⁺ CD115⁺ monocyte, CD11b⁺ CD114⁺ monocyte, CD11b⁺ CD116⁺ monocyte, CD11b⁺ CCR1⁺ monocyte, CD11b⁺ CCR2⁺ monocyte, CD11b⁺ CX3CR⁺ monocyte, CD11b⁺ CXR4⁺ monocyte, CD11b⁺ CXR6⁺ monocyte and a CD11b⁺ CD14⁺ CD33⁺ monocyte, preferably the monocyte is not a dendritic cell, which differentiation is controlled by one or more of the following transcription factors: IRF8, NFIL3, BATF3,RELB, PU/1, RBPJ, IIRF4, and/or TCF4, and more preferably is not a dendritic cell.;
(ii) the differentiated monocyte or monocyte-macrophage is differentiated by M-CSF and selected from the group consisting of a macrophage, an activated macrophage, preferably a CD11b⁺ macrophage, more preferably a CD11b⁺ CD16⁺ macrophage, CD11b⁺ CD32⁺ macrophage, CD11b⁺ CD64⁺ macrophage, CD11b⁺ CD68⁺ macrophage, preferably a CD11b⁺ CD86⁺ M1 macrophage, preferably producing inducible nitric oxide synthetase (iNOS) and/or secreting interleukin 12 (IL-12) or preferably CD11b⁺ CCR2⁺ M2 macrophage, CD11b⁺ CD204⁺ M2 macrophage, CD11b⁺ CD206⁺ M2 macrophage, CD11b⁺ CD204⁺ CD206⁺ M2 macrophage, CD11b⁺ Major Histocompatibility Complex II⁺ (MHCII) (low or hi expression) M2 macrophage, CD11b⁺ CD200R⁺ M2 macrophage, CD11b⁺ CD163⁺ M2 macrophage or activated macrophage producing and/or secreting Arginase-1 and/or interleukin 10 (IL-10); preferably the differentiated monocyte is not a foam cell expressing Lectin-like oxidized low-density lipoprotein receptor-1 (Lox1*), C-X-C chemokine receptor type 7 (CXCR7⁺) and Nuclear factor (erythroid-derived 2)-like 2 (NRF2⁺). A foam cell is a type of macrophage that localize to fatty deposits on blood vessel walls, where they ingest low-density lipoproteins and become loaded with lipids giving them a foamy appearance. These cells secrete various substances involved in plaque growth and their death promotes inflammation, thereby contributing to cardiovascular disease;
(iii) monocyte-macrophage or activated monocyte-macrophage is differentiated by M-CSF and is preferably expressing at least one chemokine receptor, preferably selected from the group consisting of CCR1, CCR2, CXCR4, and CXCR6, or at least one growth factor receptor, preferably selected from the group consisting of macrophage colony stimulating factor Receptor (CD115), granulocyte colony stimulating factor Receptor (CD114), and granulocyte-macrophage colony stimulating factor Receptor (consisting of CD116 and CD131); monocytes of these characteristics are particular suitable to treat inflammatory conditions and cancer;
(iv) the lymphocyte is selected from the group consisting of a CD3⁺ and CD4⁺ or CD8⁺ T lymphocyte, or a CD19⁺, CD20⁺, CD21⁺, CD19⁺ CD20⁺, CD19⁺ CD21⁺, CD20⁺ CD21⁺, or CD19⁺ CD20⁺ CD21⁺ B lymphocyte; or a natural killer (NK) cell, preferably the NK cell is selected from the group consisting of CD56⁺ and without CD3 expression, or CD16⁺ CD56⁺, CD56⁺ CD94⁺, CD56⁺ CD158a⁺, CD56⁺ CD158f⁺, CD56⁺ CD314⁺, CD56⁺ CD335⁺ cell; or
(v) the granulocyte is selected from the group consisting of a neutrophil, preferably a CD66b⁺ neutrophil, an eosinophil and a basophil, preferably a CD193⁺ eosinophil.

In a preferred embodiment of the isolated targeted delivery system the activated macrophage:
(i) is producible by in vitro incubation of a monocyte or macrophage or their precursors with a factor capable of altering expression markers on macrophages, preferably
   (a) with at least one M1 inducer,
   (b) with at least one M2 inducer,
   (c) or with a factor capable of altering the macrophages ability to secrete cytokines, preferably IL-10 and IL-12, chemokines and/or to produce iNOS, arginase or other immunomodulating enzymes; examples of such factors are: activated platelets, IL-4, IL-10, IL-13, immune complex of an antigen and antibody, IgG, heat activated gamma-globulin, glucocorticosteroid, tumour growth factor-β (TGF-β), IL-1R, CC-chemokine ligand 2 (CCL-2), IL-6, Macrophage colony-stimulating factor (M-CSF), peroxisome proliferator-activated receptor γ (PPARγ) agonist, leukocyte inhibitory factor (LIF), adenosine, helminth and fungal infection, lipopolysaccharide (LPS), interferon γ (INF-γ), viral and bacterial infection; in this respect it was observed that activation of a monocyte with a M1 inducer, particularly LPS will cause cell to express iNOS, that activation of a monocyte with a M1 inducer, particularly LPS will cause cell not to express Arginase-1, that activation of a monocyte with a M2 inducer, particularly IL-4 will cause cell to express Arginase-1, and that activation of a monocyte with a M2 inducer, particularly IL-4 will cause cell not to express iNOS,
(ii) is characterized by expression of at least one of following antigens: CD64, CD86, CD16, CD32, high expression of MHCII, and/or production of iNOS and/or IL-12;
(iii) is producible by in vitro incubation of a monocyte or macrophage with a factor capable of inducing the ability of the macrophage to phagocytose, e.g. IL-18, opsonins (for example complement-derived proteins such as iC3b, immunoglobulin G), calcitonin gene-related peptide (CGRP), lipopolysaccharide (LPS), interferon γ (INF-γ), viral infection and/or bacterial infection;
(iv) is characterized by expression of at least one of following antigens: CD204, CD206, CD200R; CCR2, transferrin receptor (TfR), CXC-motive chemokine receptor 4 (CXCR4), CD163, and/or T cell immunoglobulin-domain and mucin-domain 2 (TIM-2), and/or show low expression of MHCII; activated macrophages having these properties are particularly suitable for complexes comprising ferritin as the iron binding protein;
(v) has the ability to phagocytose; and/or
(vi) is capable of cytokine secretion, preferably of IL-12, or IL-10, or production of inducible nitric oxide synthetase (iNOS) (or other pro-inflammatory compounds), arginase or other immunosuppressive/anti-inflammatory compounds.

In a preferred embodiment of the isolated targeted delivery system the M1 inducer for differentiating macrophages into M1 macrophages is selected from the group consisting of lipopolysaccharide (LPS), interferon γ (INF-γ), and viral and bacterial infection and the M2 inducer for differentiating macrophages into M2 macrophages is selected from the group consisting of IL-4, IL-10, IL-13, immune complex of an antigen and antibody, IgG, heat activated gamma-globulin, glucocorticosteroid, tumour growth factor-β (TGF-β), IL-1R, CC-chemokine ligand 2 (CCL-2), IL-6, Macrophage colony-stimulating factor (M-CSF), peroxisome proliferator-activated receptor γ (PPARγ) agonist, leukocyte inhibitory factor (LIF), adenosine, helminth and fungal infection.

In a preferred embodiment of the targeted delivery system of the present invention the monocyte-macrophage:
(i) is producible from a CD34⁺ hematopoietic precursor cell;
(ii) is producible by in vitro incubation of monocytes with at least one inducer, preferably M1 or M2 inducer, more preferably at least one M2 inducer;
(iii) is characterized by expression of at least one of the following antigens: TfR, CD163, TIM-2, CD14, CD16, CD33, and/or CD115;
(iv) is characterized by expression of at least one of the following antigens: TfR, CD163, TIM-2, CXCR4, CD14, and/or CD16; and/or
(v) has the ability to phagocytose; and/or
(vi) is not a dendritic cell which differentiation is controlled by one or more of the following transcription factors: IRF8, NFIL3, BATF3 or RELB, PU/1, RBPJ, IRF4 or TCF4.
   In this embodiment of the targeted delivery system of the present invention the M1 inducer for differentiating monocyte-macrophage cells is selected from the group consisting of LPS, INF-γ or viral or bacterial infection or the M2 inducer for differentiating monocytes is selected from the group consisting of IL-4, IL-10, IL-13, immune complex of an antigen and antibody, IgG, heat activated gamma-globulins, Glucocorticosteroids, TGF-β, IL-1R, CCL-2, IL-6, M-CSF, PPARγ agonist, Leukocyte inhibitory factor (LIF), cancer-conditioned medium, cancer cells, adenosine and helminth or fungal infection.

In a preferred embodiment of the targeted delivery system of the present invention the lymphocyte:
(i) is obtainable from blood, spleen, or bone marrow or is producible from a CD34⁺ precursor cell as known to the skilled person and also described in the, e.g. Lefort and Kim, 2010, J Vis Exp 40: 2017; Tassone and Fidler, 2012, Methods in Molecular Biology 882: 351-357; Kouro et al. 2005, Current Protocols in Immunology, 66:F22F.1:22F.1.1-22F.1.9.;
(ii) is an immunologically competent lymphocyte;
(iii) expresses antigen specific T cell receptors; and/or
(iv) is characterized by expression of at least one of the following antigens: (a) CD3 and CD4 or CD8 or (b): CD19, CD20, CD21, CD19 CD20, CD19 CD21, CD20 CD21, or CD19 CD20 CD21 antigen, and is preferably capable of producing immunoglobulins

In a particularly preferred embodiment the CD45+ lymphocytes is a NK cell, which
(i) is obtainable from blood, spleen or bone marrow or producible from a CD34⁺ precursor cell; and/or
(ii) is characterized by the lack of CD3 expression and expression of at least one of the following CD56⁺ and/or CD94⁺, CD158a⁺ CD158f⁺ CD314⁺ CD335⁺.

In a preferred embodiment of the targeted delivery system of the present invention the granulocyte:
(i) is obtainable from blood, spleen or bone marrow or producible from a CD34⁺ precursor cell as described, e.g. in Kuhs et al. 2015, CurrProtocImmunol 111:7.23-1-7.23.16; Coquery et al. 2012, Cytometry A 81(9): 806-814; Swemydas and Lionakis 2013, J Vis Exp 77: 50586.;
(ii) is characterized by expression of at least one of the following CD66b and/or CD193;
(iii) is a polymorphonuclear leukocyte characterized by the presence of granules in its cytoplasm; and/or
(iv) is characterized by expression of at least one of the following: TfR, CD163, TIM-2, and/or CXCR4.

The cell comprised in the isolated targeted delivery system may also be a mesenchymal stem cell. The term “mesenchymal stem cell” or “MSC” is used in the context of the present invention to refer to adult stem cells which are non-haematopoietic, multipotent stem cells with the capacity to differentiate into mesodermal lineage such as osteocytes, adipocytes and chondrocytes as well ectodermal (neurocytes) and endodermal lineages (hepatocytes). MSCs express cell surface markers like cluster of differentiation (CD)73, CD90, and CD105 and lack the expression of CD45, CD34, CD14/CD11b, CD19/CD20/CD79a, and HLA (human leucocyte antigen)-DR. Human MSCs for the first time were reported in the bone marrow and till now they have been isolated from various tissues, including adipose tissue, placenta, amniotic fluid, endometrium, dental tissues, umbilical cord blood and umbilical cord tissue (Wharton's jelly). They also have been derived (i.e. differentiated) from Induced Pluripotent Stem Cells (iPSCs). Thus, in a preferred embodiment of the invention, the MSC is selected from the group consisting of an umbilical cord MSC, a bone marrow MSC, an adipose MSC, a placenta MSC, a dental MSC, an amniotic fluid MSC, an endometrium MSC, and an iPSC-derived MSC. Preferably, it is an umbilical cord blood MSC or an umbilical cord tissue (or Wharton's jelly) MSC. Furthermore, it is preferred that the MSC is a human MSC. The present inventors have observed that such MSCs stem cells can be loaded with the complex according to the invention and deliver it into cancer cells.

In a seventh aspect the present invention relates to pharmaceutical or diagnostic composition comprising the polypeptide of the first aspect, the conjugate of the second aspect, the complex of the third aspect or the isolated targeted delivery system of the fourth aspect and a pharmaceutically acceptable carrier and/or suitable excipient(s).

In instances where the pharmaceutical or diagnostic composition comprises living cells, it is preferred that carriers and excipients are chosen such as to keep the cells alive.

“Pharmaceutically acceptable” means approved by a regulatory agency of the Federal or a state government or listed in the U.S. Pharmacopeia or other generally recognized pharmacopeia for use in animals, and more particularly in humans.

The term “carrier”, as used herein, refers to a pharmacologically inactive substance such as but not limited to a diluent, excipient, surfactants, stabilizers, physiological buffer solutions or vehicles with which the pharmaceutically active substance is administered. Such pharmaceutical carriers can be liquid or solid. Liquid carrier include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like.

“Surfactants” include anionic, cationic, and non-ionic surfactants such as but not limited to sodium deoxycholate, sodium dodecylsulfate, Triton X-100, and polysorbates such as polysorbate 20, polysorbate 40, polysorbate 60, polysorbate 65 and polysorbate 80.

“Stabilizers” include but are not limited to mannitol, sucrose, trehalose, albumin, as well as protease and/or nuclease antagonists.

“Physiological buffer solution” include but are not limited to sodium chloride solution, demineralized water, as well as suitable organic or inorganic buffer solutions such as but not limited to phosphate buffer, citrate buffer, tris buffer (tris(hydroxymethyl)aminomethane), HEPES buffer ([4 (2 hydroxyethyl)piperazino]ethanesulphonic acid) or MOPS buffer (3 morpholino-1 propanesulphonic acid). The choice of the respective buffer in general depends on the desired buffer molarity. Phosphate buffer are suitable, for example, for injection and infusion solutions.

The term “adjuvant” refers to agents that augment, stimulate, activate, potentiate, or modulate the immune response to the pharmaceutically active substance comprised in the composition at either the cellular or humoral level, e.g. immunologic adjuvants stimulate the response of the immune system to the actual antigen, but have no immunological effect themselves. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-10, IL-2, IL-7, IL-12, IL-18, GM-CFS, and INF-7) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g. polyarginine or polylysine).

As indicated above, the term “CD45⁺ leukocyte cell” is used throughout this specification to refer to a CD45⁺ monocyte, CD45⁺ monocyte-macrophage, CD45⁺ lymphocyte and/or CD45⁺. Preferably, the monocyte is not a dendritic cell, which differentiation is controlled by one or more of the following transcription factors: IRF8, NFIL3, BATF3, RELB, PU/1, RBPJ, IIRF4, and/or TCF4, and more preferably is not a dendritic cell. Preferred subpopulations in these general categories of leukocytes are defined in the following by structural parameters, e.g. presence or absence of a given protein, functional properties and/or method of their production/differentiation. As outlined above, the targeted delivery system of the present invention still provides the advantages outlined above, if in a population of cells not every cell has a particular property in as long as the majority of cells within that population has that property. Thus, in the following the property of one preferred cell of the targeted delivery system of the present invention is described. It is appreciated by the skilled person that a pharmaceutical composition of the present invention will comprises millions of cells and that not every cell within the population will have the functional and/or structural properties outlined herein but that the pharmaceutical composition can nevertheless be used to treat a disease, if the majority of cells share the respective functional and/or structural properties.

The cells comprised in the targeted delivery system, in particular CD45⁺ leukocyte cells or MSCs, originate from the patient to be treated. In such case the cell loaded with the complex would be autologous to the patient. It is also envisioned that patients are MHC typed prior to treatment with the targeted delivery system of the present invention and that the cell type used for a given patient is MHC matched to the patient. In these two preferred embodiments the cell is a primary cell or derived by a low number of differentiation steps from a primary cell. Alternatively, the cell may be from an immortalized but preferably non-transformed cell line.

The blood used for isolation of CD45⁺ leukocyte cells, i.e. CD45⁺ monocyte, CD45⁺ monocyte-macrophage, CD45⁺ granulocyte, or CD45⁺ lymphocyte, in particular CD45⁺ NK cell, is preferably obtained from the patient to be treated or from a healthy donor. Alternatively the blood can be obtained from the blood bank. Use of umbilical cord blood is also considered herein.

In an eight aspect the present invention relates to the polypeptide of the first aspect, the conjugate of the second aspect, the complex of the third aspect or the isolated targeted delivery system of the fifth aspect for use in medicine.

In a ninth aspect the present invention relates to the polypeptide of the first aspect, the conjugate of the second aspect, the complex of the third aspect or the isolated targeted delivery system of the fifth aspect for use in treating, preventing and diagnosing a tumour, preferably a solid tumour and/or its metastases, preferably breast cancer, pancreatic cancer, bladder cancer, lung cancer, colon cancer, or a tumour having hypoxic areas; an inflammatory disease or ischemic areas, in particular in skin wounds or after organ infarctus (heart) or ischemic retina; or for prophylactic or therapeutic vaccination, in particular to prevent or treat an infectious disease or cancer. This aspect also includes targeted delivery of antigens to physiological or non-physiological lymph nodes in order to vaccinate an individual or to induce immune memory.

In a tenth aspect the present invention relates to method of treating, preventing or diagnosing a tumour, preferably a solid tumour and/or its metastases, preferably breast cancer, pancreatic cancer, bladder cancer, lung cancer, colon cancer, ovarian cancer, liver cancer, glioma/glioblastoma or a tumour having hypoxic areas; an inflammatory disease or ischemic areas, in particular in skin wounds or after organ infarctus (heart) or ischemic retina; or a method of prophylactic or therapeutic vaccination, in particular to prevent or treat an infectious disease or cancer by administering an effective amount of the polypeptide of the first aspect, the conjugate of the second aspect, the complex of the third aspect or the isolated targeted delivery system of the fifth aspect to a subject in need thereof. This aspect also includes targeted delivery of antigens to physiological or non-physiological lymph nodes in order to vaccinate an individual or to induce immune memory.

The term “treatment” as used herein includes all types of preventive and/or therapeutic interventions medically allowed for the purpose of cure, temporary remission, prevention, etc. for different purposes including delaying or stopping the progress of a disease, making a lesion regress or disappear, preventing onset, or inhibiting recurrence.

### BRIEF DESCRIPTION OF DRAWINGS

FIG. 1: In silico analysis of mutants: Hot spot prediction results by using PredHS2for the human H-Ferritin-TfR1 complex and FoldX for the human H-Ferritin-DNA virtual complex. True positives, common for both DNA binding and TfR1 binding are represented as CPK.

FIG. 2: Sensorgrams corresponding to the interaction between the immobilized his-tagged TFRC receptor and human ferritin. Panel A: mutant Q11E, panel B: wild type. X-axis: time (s). The same amount of receptor was trapped onto the chip surface (see methods section for details). Five different analyte concentrations (0.0625, 0.125, 0.250, 0.5 and 1 mg/ml) were used. For all analyte concentrations measured, the amount of ferritin bound is higher for the Q11E mutant than for wild-type ferritin. The global fit using a simple 1:1 binding mode indicates a higher affinity and correspondingly a lower K_{D} value.

FIG. 3: Native gel analysis of wild type ferritin and mutants showing that wild type ferritin mainly forms of double glued frames, aggregates of cages and larger forms of aggregates, however, mutations decrease the ability of ferritin to form aggregates, therefore the mutant ferritin variants are present as 24-mers (homogeneous cages).
Lane 1: Ferritin wild type-dox concentrated on 10 kDa Amicon.
Lane 2: Ferritin wild type-dox concentrated on 100 kDa Amicon
Lane 3: Q11E mutant-dox concentrated on 10 kDa Amicon.
Lane 4: Q11E mutant-dox concentrated on 100 kDa Amicon.
Lane 5: Q11E-Q15E mutant-dox concentrated on 10 kDa Amicon.
Lane 6: Q11E-Q15E mutant-dox concentrated on 100 kDa Amicon.

FIG. 4: Native gel analysis of wild type ferritin and mutants showing that storage conditions did not adversely affect the stability of the cages in the mutants, and the mutant ferritin variants are still present as 24-mers (homogeneous cages) after storage.
Lane 1: Q11E mutant-dox concentrated on 10 kDa Amicon.
Lane 2: Q11E mutant-dox concentrated on 100 kDa Amicon.
Lane 3: Q11E-Q15E mutant-dox concentrated on 10 kDa Amicon.
Lane 4: Q11E-Q15E mutant-dox concentrated on 100 kDa Amicon.
Lane 5: Ferritin wild type-dox concentrated on 10 kDa Amicon.
Lane 6: Ferritin wild type-dox concentrated on 100 kDa Amicon.

FIG. 5: Graphical representation of the calculation of doxorubicin loading efficiency for Ft wild type and Ft mutants. The average particle numbers per cage along with the median are marked on the graph.

FIG. 6: UV-Vis spectrum of Ft wild type and Q11E mutant after doxorubicin encapsulation. The initial concentration for both proteins was the same and equal 28.5 mg/ml. The final concentrations for Ft wild type and Q11E mutant were 10.2 mg/ml and 28.5 mg/ml. Each concentration and the recorded spectra are for a volume of 1 ml protein solution. The extinction coefficient for Ft wild type and Q11E mutant are the same and equal 18600 M⁻¹ cm⁻¹ (λ=278 nm).

FIG. 7: Graphs of tumour cell viability after 72 h co-culture with macrophages. The concentration of ferritin cages filled with doxorubicin was the same for each variant of ferritin and was equal to 1 mg/ml.

FIG. 8: Picture of gel showing RNA association with Q11E ferritin mutant that is not observed in case of wild-type protein (wt).

FIG. 9. The LC-MS spectrum of ferritin after conjugation with vcMMAE. The spectrum shows that it is mostly a ferritin fraction with two drug molecules attached. The molar drug to protein ratio of the conjugate is equal to 1.95.

### EXAMPLE SECTION

### Example 1—In Silico Analysis of Mutants

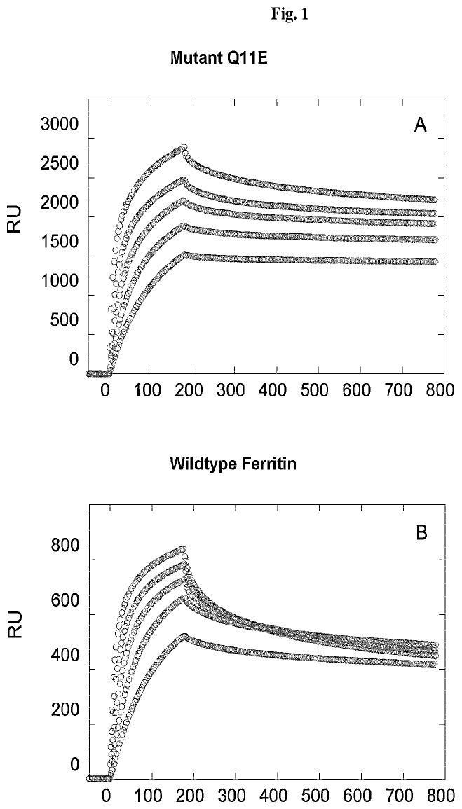

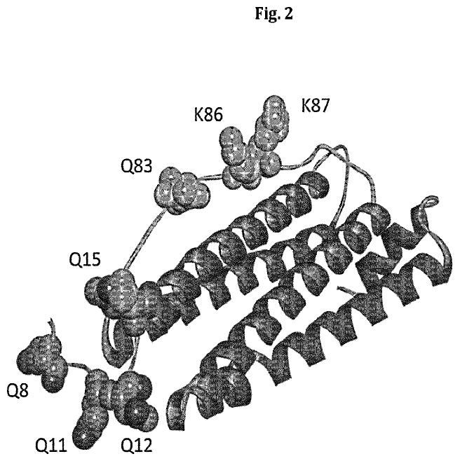

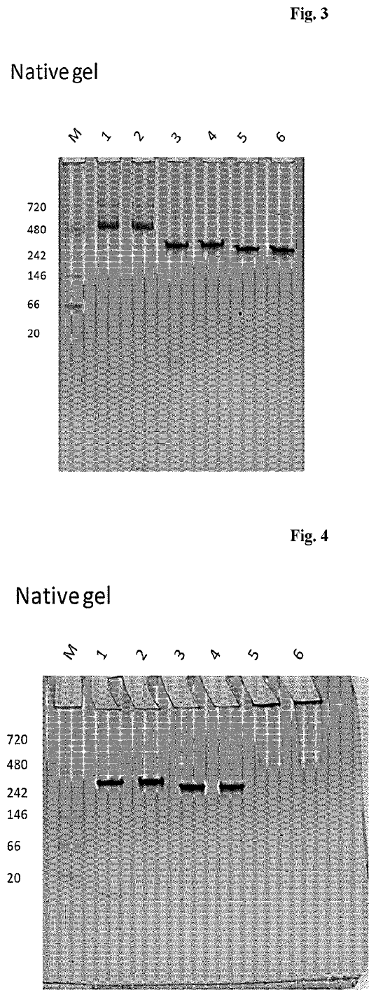

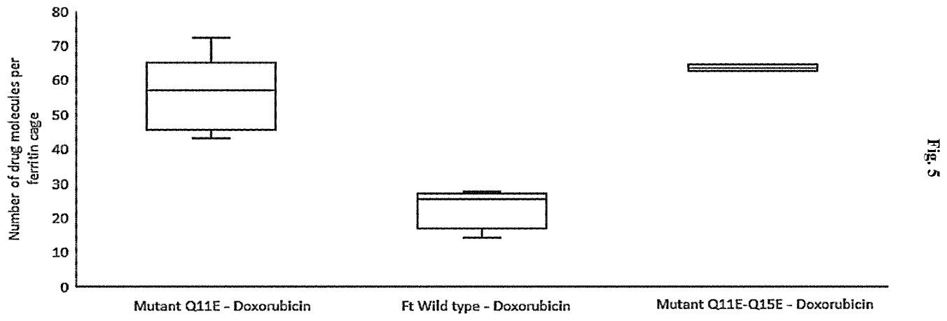

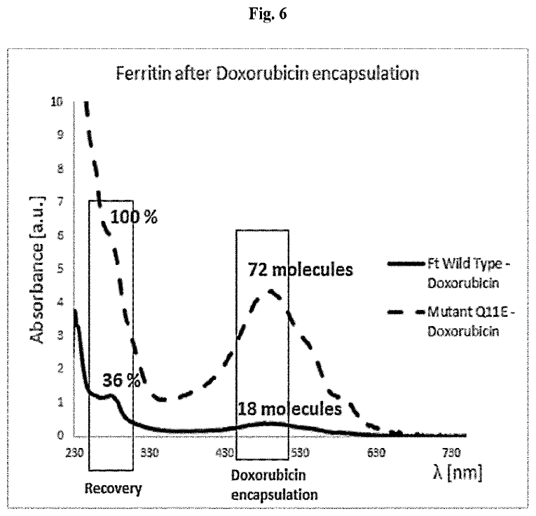

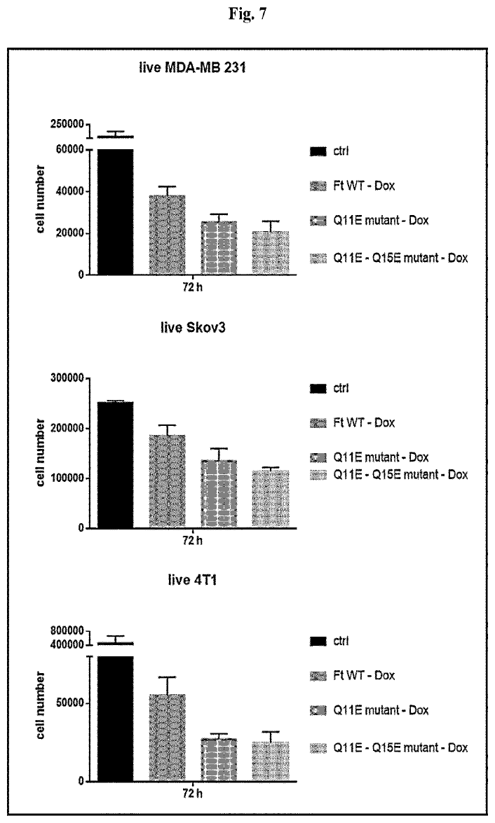

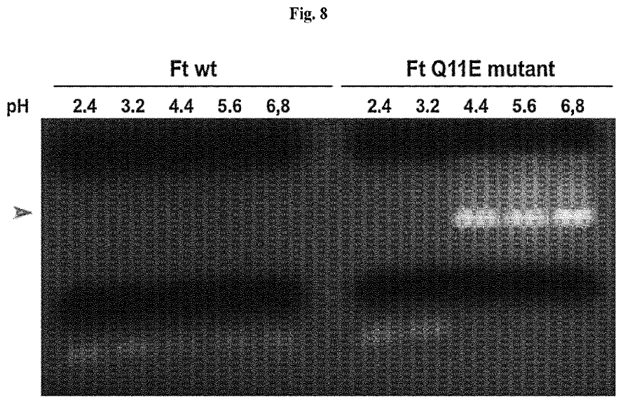

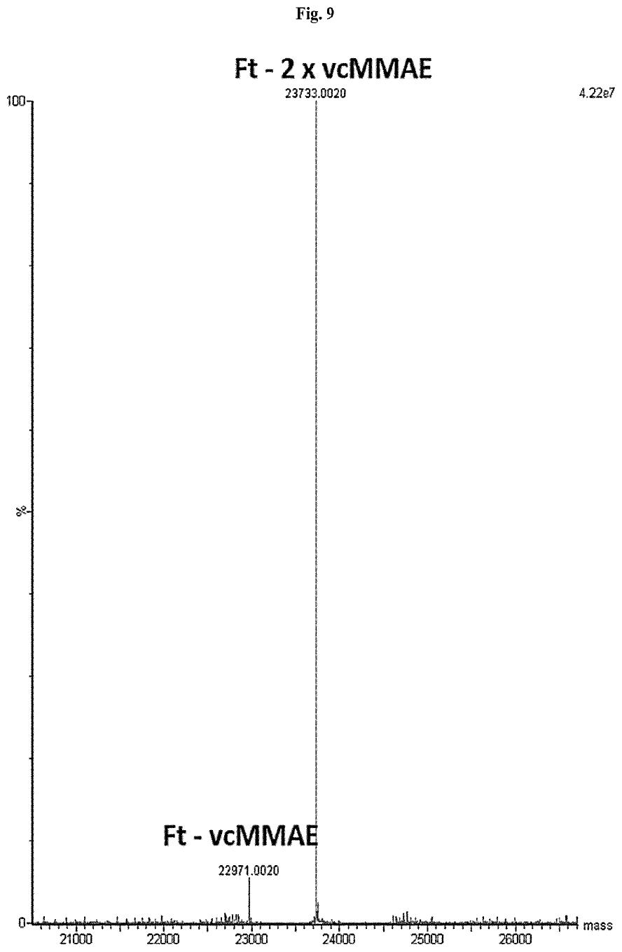

Identification of small portions of a protein-protein interface that contribute to the majority of the binding free energy can provide crucial information for understanding the nature of the interaction and recognition properties. These portions are referred to as “hot spots” in recent computational chemistry approaches (Hao Wang, et al., Sci. Rep. 8, 14285 2018). Here, the inventors describe the application of the PredHS2 software (http://predhs2.denglab.org) coupled to MD minimization in order to predict hot spots from the complex of human H chain ferritin and Transferrin receptor (PDB ID:6H5I). Based on PredHSmethod (Wei, L, et Al. Comb. chemistry & high throughput screening 19, 144-152 2016), the inventors built a dataset of 14 interface residues on H Ferritin interface that corresponds to the contacts obtained from the CD71/H-ferritin complex recently identified by Montemiglio et al., (Montemiglio et al., 2019 Nat Comm 10 1121-1121). Then the inventors generated a set of 476 sequences (single mutants of the 14 positions) obtained after removal of redundant and irrelevant sequences utilizing a two-step feature selection method, which consists of a minimum Redundancy Maximum Relevance (mRMR) procedure and a sequential forward selection process that eliminates all mutations that are considered not compatible with folding properties (e.g. Gly or Pro within alpha helices regions). Thereafter, energy minimization of the relevant structure, exposure to solvent and energy features, together with Euclidean and Voronoi neighbourhood properties was carried out. In order to assess the performance of the prediction model, the inventors adopted 10-fold cross-validation together with commonly used measures, such as specificity (SPE), precision (PRE), sensitivity (SEN/Recall), accuracy (ACC), F1-score (F1) and Matthews correlation coefficient (MCC). Data relative to the best energy matches (ZAPP) and measures are listed in Table 1. As apparent, the isosteric glutamine substitutions with a glutamate residue predicted a higher binding free energy contribution whereas non-isosteric mutations (even when bearing the same charge) invariably led to a decrease in binding free energy contribution (RMSE). Apparently, Voronoi contribution (polynomial squared distances minimization), played a key role in contributing to the binding free energy as it confers energy penalties to the voids generated by missing atoms in non-isosteric mutants, or even higher gaps in the case of bulkier residues. As shown in FIG. 1, four hot spots (8, 11, 12 and 15) have been experimentally determined at the binding interface. These residues were individually taken into account. Multiple mutants have not been considered in these calculations as the resulting binding free energies appeared to be unrealistically high.

TABLE I
Prediction of “hot spots” for the complex of human
H chain ferritin and Transferrin receptor.
The four residues in positions 8, 11, 12 and 15 (PDB ID: 6H5I)
were found to be the most important contributors to the
H-ferritin/CD71 receptor complex formation. Here the inventors
show that individual, isosteric mutations of these “hot spots”
provide a further binding free energy gain to the complex.

| | Composition of ZAPP and Performance | | | | | | |
|---|---|---|---|---|---|---|---|
| | of Energy Functions with Terms | | | | | | |
| | RMSE | | | | | | |
| | (Kcal | | | | | | |
| Mutant | mol⁻¹) | ACC | SPE | PRE | SEN | F1 | MCC |
| Q8E | 7.2 | 0.89 | 0.91 | 0.85 | 0.78 | 0.79 | 0.70 |
| Q11E | 8.1 | 0.87 | 0.95 | 0.84 | 0.83 | 0. 80 | 0.70 |
| N12D | 4.9 | 0.91 | 0.93 | 0.85 | 0.81 | 0.79 | 0.70 |
| Q15E | 5.3 | 0.94 | 0.94 | 0.86 | 0.87 | 0.82 | 0.70 |
| WT | 4.3 | 0.88 | 0.94 | 0.86 | 0.79 | 0.80 | 0.70 |

A second algorithm has been applied based on Protein-assisted DNA assembly (PADA1) algorithm to predict and model the binding of double-stranded DNA (dsDNA) to proteins. PADA1 includes an empirical interaction model generator in combination with an ultra-fast statistical knowledge-based force field, which act in synergy in order to perform dsDP docking (Blanco J D, et al., Nucleic Acids Res. 2018 May 4; 46(8):3852-3863).This algorithm uses fragment pairs (peptide paired to short dsDNA) that represent empirical, compatible backbone conformations found in nature. DNA-protein structures modeled by PADA1 have been used in combination with FoldX (protein design software) to predict DNA recognition sequences. The cooperative action between PADA1 and FoldX, for side chain refinement and interface optimization, turns ModelX in a powerful modelling tool for predicting key residues at the core of ferritin-DNA interaction. In the case of Human H ferritin, we measured the atomic “all-to-all” distances between the protein fragment and the corresponding dnaX fragment. Then, using the atomic distance distributions, we obtained the statistical parameters (mean and standard deviation of the distances) for all possible contacts between the protein and dsDNA fragments included in the interaction database. All-to-all distances between contacting nucleotide-amino acid pairs were measured according to the limits suggested by Blanco J D et al., (a contact is considered when at least one atom of the amino acid, including side chains, is less than 4 Å from any atom in the nucleotide). Most interestingly, 7 residues were found to be responsible for nucleic acid binding properties, which comprise the 4 glutamines already demonstrated to contribute to the receptor binding interface plus glutamine 83 together with lysines 86 and 87 (see FIG. 1).

### Example 2—In Vitro Binding to Ferritin Receptor

Binding of wild type and Q11E mutant ferritin to TfR1 was analysed by surface plasmon resonance.

### Examples 3-8

The inventors generated further ferritin variants based on the Q11E and Q11E-Q15E mutants by adding the mutations K54E, K72E, K87Q, K144E, C91S and C103S. Mutation C131S was further added to mutant Q11E. The properties of these ferritin variants were further analysed in examples 3-8.

### Example 3

Native PAGE gel has been performed in order to check the size of the protein after encapsulation and purification. It clearly shows that wild type protein shows a heterogeneity of forms, in addition to cages it contains aggregates of cages and larger forms of aggregates, in contrary to mutated protein, which is are present as 24-mers (homogeneous cages). A greater degree of aggregation adversely affects the loading efficiency and protein recovery after loading with doxorubicin (FIG. 3).

### Example 4

Storage conditions for wild type ferritin are limited. Storage in a freezer (−80 degrees Celsius) and thawing causes an increase of cages aggregation, which prevents their separation on native electrophoresis. In contrast, the Q11E mutant reduces the presence of aggregates and allows the storage of ferritin cages with doxorubicin in the freezer (FIG. 4).

### Example 5

The calculation of the encapsulation efficiency indicates that Ft mutants Q11E and Q11E-Q15E are able to load on average more doxorubicin molecules into their cages compared to Ft wild type, 55, 63, 23 molecules, respectively (FIG. 5).

### Example 6

UV-Vis spectrum analysis has shown that protein recovery after loading was 100% for mutant Q11E and only 38% for wild ferritin (FIG. 6).

### Example 7

In vitro experiments have shown that ferritin mutants reveal better cytotoxicity against tumor cells.

The doxorubicin packed proteins were inserted into macrophages and then co-cultured with breast and ovarian cancer cell lines: MDA-MB 231, Skov3 and 4T1. The number of viable cells after co-culture was the lowest for the Q11E-Q15E mutant (FIG. 7).

### Example 8

1 ml reaction containing 2 mg Ft and 4 ug siRNA in DPBS, pH lowered to values as indicated (2.5, 3.2, 4.4, 5.6 and 6.8, respectively). Samples were incubated 15 min in RT, then NaOH was added to adjust pH to neutral (pH 7). Non-associated free siRNA was removed by 4 centrifugation steps on Amicons with 100 kDa cut-off. As shown in FIG. 8, Ferritin Q11E mutant associated with siRNA when incubated in pH range 4.4 to 6.8 (arrow), but not in 2.4 and 3.2, conditions in which Ferritin cage is likely to be disrupted. This observation suggests that Ft nanocage integrity might be crucial for observed association. In contrast, no association with siRNA was observed for wild type Ferritin variant, independently from pH conditions during Ft incubation with siRNA.

### Example 9

Human heavy chain ferritin can be covalently linked to host hydrophobic drug molecules within the cysteine residues. Maleimide functionalized drugs, such as a tubulin inhibitor Monomethyl Auristatin (MMAE) is one of the most notable examples of potent cytotoxic that can be readily and specifically attached. The inventors have conjugated this drug to ferritin according to the following procedure: The auristatin E analogue, maleimidocaproyl-valine-citrulline-p-aminobenzoyloxycarbonyl-monomethyl auristatin E (vcMMAE) was obtained from MedChem Express (Princeton, NJ). The ferritin vcMMAE adduct was prepared as follows: Human heavy chain ferritin according to SEQ ID NO: 77 was used. Ferritin solution was adjusted to a concentration of 125 μM with reaction buffer (20 mM HEPES ((4-(2-hydroxyethyl)-1-piperazineethanesulfonic acid)), 0.04% Polysorbate 80, pH 7.0) and conjugated with 5-fold molar excess of vcMMAE at room temperature at 4° C. for 4 hours. Maleimide groups react efficiently and specifically with free (reduced) sulfhydryls at pH 6.5-7.5 to form stable thioether bonds. The final conjugate was dialyzed in washing buffer (20 mM HEPES ((4-(2-hydroxyethyl)-1-piperazineethanesulfonic acid)), 0.02% Polysorbate 80, 2% Glycerin, pH 7.0), to remove unbound vcMMAE, and concentrated at Amicon® centrifugal filter device. The molar drug to protein ratio of the obtained conjugate was determined by LC-MS analysis and it was equal to 1.95 (see FIG. 9). The concentration of Ft-vcMMAE conjugate was determined by BCA colorimetric assay based on the absorbance at 562 nm.

### Items

1. A ferritin variant polypeptide, wherein one or more cysteine residues, in particular cysteine residues at position 91, 103 and/or 131 indicated with respect to SEQ ID NO. 1, are deleted or substituted, preferably substituted with serine residues.
2. The ferritin variant polypeptide of item 1, wherein the ferritin variant polypeptide has a sequence according to SEQ ID NO. 82, SEQ ID NO. 1 or SEQ ID NO. 2, wherein at least one, preferably all, cysteine residues at position 91, 103 and/or 131 are deleted or substituted, preferably substituted with serine residues, and wherein the sequences according to SEQ ID NO. 82, SEQ ID NO. 1 and SEQ ID NO. 2 may further comprise 1-5, 1-10, 1-15, 1-20 or 1-25 amino acid mutations outside position 91, 103 and/or 131.
3. The ferritin variant polypeptide of item 1 or 2, wherein one, two, three or four, preferably four, lysine residues, preferably lysine residues at position 54, 72, 87 and/or 144 indicated with respect to SEQ ID NO. 1 (human wild-type heavy chain ferritin), are deleted or substituted with a non-basic amino acid, preferably E or Q, most preferably wherein K54 is substituted with E, K72 is substituted with E, K87 is substituted with Q and K144 is substituted with E.
4. The ferritin variant polypeptide of any one of items 1 to 3, wherein the ferritin variant polypeptide has a sequence according to SEQ ID NO. 75 or SEQ ID NO. 76 or a sequence according to SEQ ID NO. 75 or SEQ ID NO. 76 comprising 1-5, e.g. 1, 2, 3, 4 or 5, or 1-10, e.g. 1, 2, 3, 4, 5, 6, 7, 8, 9 or 10 amino acid mutations outside position 91, 103 and/or 131.
5. The ferritin variant polypeptide of any one of items 1 to 4, further comprising a transferrin receptor binding domain (TRBD) of a ferritin variant wherein the TRBD in comparison to the wild-type ferritin on which it is based comprises one or more glutamine residues mutated into glutamic acid residues and/or one or more asparagine residues mutated into aspartic acid residues, wherein in particular at least one, preferably all mutations are comprised in the 20 N-terminal amino acids of the wild-type ferritin.
6. The ferritin variant polypeptide of item 5, wherein the TRBD comprises at least the following amino acid sequence:

(SEQ ID NO. 3)
MTTASX₁SZVRZBYHZDX₂EAA

X₁=S or T, preferably T;
X₂=S or A, preferably S;
Z=Q or E; and
B=N or D;
wherein at least one Z or B is E or D,
which may further comprise one, two or three amino acid substitutions outside Z and/or B, and wherein the M at position 1 may be present or absent.
7. The ferritin variant polypeptide according to item 5 or 6, wherein the TRBD comprises at least an amino acid sequence selected from the group comprising SEQ ID NO. 04 to SEQ ID NO. 63, particularly from the group consisting of SEQ ID NO. 05, 11, 12, 15, 20, 26, 27, 30, 35, 41, 42, 45, 50, 56, 57 and 60, more particularly from the group consisting of SEQ ID NO. 05, 12, 20, 27, 35, 42, 50 and 57, which may further comprise one, two or three amino acid substitutions outside amino acid positions 8, 11, 12 and/or 15, and wherein the M at position 1 may be present or absent.
8. The ferritin variant polypeptide according to any of items 5 to 7, wherein the affinity of the TRBD to TfR-1 is increased in comparison to the TRBD of the wild-type ferritin at least (≥) 1.5×, ≥2×, ≥3×, ≥4×, ≥5×, ≥10×, ≥20×, ≥30×, ≥40×, ≥50×, but less than (≤) 100×, ≤75×≤50×, ≤40×, ≤30×, ≤20×, ≤10×, or ≤5×, in particular the affinity of the TRBD to TfR-1 is increased between 1.5×-50×, 2×-50×, 3×-50×, 4×-50×, 5×-50×, 10×-50×, 20×-50×, 30×-50×, 40×-50×, 1.5×-10×, 2×-20× or 5×-30× in comparison to the TRBD of the wild-type ferritin.
9. A nucleic acid encoding the polypeptide of any of items 1 to 8.
10. A vector comprising the nucleic acid of item 9.
11. A conjugate comprising the polypeptide of items 1 to 8 and at least one label and/or at least one drug.
12. A complex comprising at least one polypeptide of items 1 to 8 and/or at least one conjugate of item 16.
13. The complex of item 12 further comprising at least one label and/or at least one drug.
14. The conjugate of item 11 or the complex of items 12 or 13, wherein the label is selected from the group consisting of
   a. a fluorescent dye, in particular a fluorescent dye selected from the group consisting of the following classes of fluorescent dyes: Xanthens, Acridines, Oxazines, Cynines, Styryl dyes, Coumarines, Porphines, Metal-Ligand-Complexes, Fluorescent proteins, Nanocrystals, Perylenes and Phtalocyanines as well as conjugates and combinations of these classes of dyes;
   b. a radioisotope/fluorescence emitting isotope, in particular a radioisotope/fluorescence emitting isotope selected from the group consisting of alpha radiation emitting isotopes, gamma radiation emitting isotopes, Auger electron emitting isotopes, X-ray emitting isotopes, fluorescent isotopes, such as 65Tb, fluorescence emitting isotopes, such as 18F, 51Cr, 67Ga, 68Ga, 89Zr, 111In, 99mTc, 140La, 175Yb, 153Sm, 166Ho, 88Y, 90Y, 149Pm, 177Lu, 47Sc, 142Pr, 159Gd, 212Bi, 72As, 72Se, 97Ru, 109Pd, 105Rh, 101m15Rh, 119Sb, 128Ba, 123I, 124I, 131I, 197Hg, 211At, 169Eu, 203Pb, 212Pb, 64Cu, 67Cu, 188Re, 186Re, 198Au and 199Ag as well as conjugates and combinations of above with proteins, peptides, small molecular inhibitors, antibodies or other compounds;
   c. a detectable polypeptide, in particular an autofluorescent protein, preferably green fluorescent protein or any structural variant thereof with an altered adsorption and/or emission spectrum or nucleic acid encoding a detectable polypeptide; and
   d. a contrast agent, in particular a contrast agent comprising a paramagnetic agent, preferably selected from Gd, Eu, W and Mn, or ferrihydride.
15. The conjugate of item 11 or 14 or the complex of items 12 to 13, wherein the drug is selected from the group consisting of an anticancer drug, in particular a cytostatic drug, cytotoxic drug or prodrug thereof, an anti-arteriosclerotic drug, and an anti-inflammatory or immunomodulatory drug.
16. The conjugate of item 11, 14 or 15 comprising a drug, wherein the drug is auristatin, in particular monomethyl auristatin (MMAE), conjugated to the polypeptide via a maleimidocaproyl-valine-citrulline-p-aminobenzoyloxycarbonyl linker.
17. An isolated targeted delivery system comprising a cell, wherein the cell comprises the polypeptide of items 1 to 8, the conjugate of item 11 or 14 to 16, or the complex of item 12 to 16, wherein particularly the cell is a CD45+ leukocyte, more particularly a CD45+ leukocyte selected from the group consisting of a monocyte, a differentiated monocyte, lymphocyte and a granulocyte.
18. A pharmaceutical or diagnostic composition comprising the polypeptide of items 1 to 8, the conjugate of item 11 or 14 to 16 or the complex of item 12 to 16 or the isolated targeted delivery system of item 17 and a pharmaceutically acceptable carrier and/or suitable excipient(s).
19. The polypeptide of items 1 to 8, the conjugate of item 11 or 14 to 16 or the complex of item 12 to 16 or the isolated targeted delivery system of item 17 for use in medicine.

1. A ferritin variant polypeptide, wherein at least one, at least two, at least three or at least four, preferably four, lysine residues, preferably lysine residues at position 54, 72, 87 and/or 144 indicated with respect to SEQ ID NO. 1 (human wild-type heavy chain ferritin), are deleted or substituted with a non-basic amino acid, preferably E or Q.

2. The ferritin variant polypeptide of claim 1, wherein a lysine residue at position 54 indicated with respect to SEQ ID NO. 1 is deleted or substituted with a non-basic amino acid.

3. The ferritin variant polypeptide of claim 1, wherein a lysine residue at position 72 indicated with respect to SEQ ID NO. 1 is deleted or substituted with a non-basic amino acid.

4. The ferritin variant polypeptide of claim 1, wherein lysine residues at position 54 and 72 indicated with respect to SEQ ID NO. 1 are deleted or substituted with a non-basic amino acid.

5. The ferritin variant polypeptide of claim 1, wherein lysine residues at position 54, 72 and 87 indicated with respect to SEQ ID NO. 1 are deleted or substituted with a non-basic amino acid.

6. The ferritin variant polypeptide of claim 1, wherein lysine residues at position 54, 72 and 144 indicated with respect to SEQ ID NO. 1 are deleted or substituted with a non-basic amino acid.

7. The ferritin variant polypeptide of claim 1 or 2, wherein lysine residues at position 54, 72, 87 and 144 indicated with respect to SEQ ID NO. 1, are deleted or substituted with a non-basic amino acid.

8. The ferritin variant polypeptide of any one of claims 1 to 7, wherein K54 is substituted with E, K72 is substituted with E, K87 is substituted with Q and K144 is substituted with E.

9. The ferritin variant polypeptide of any one of claims 1 to 8, wherein the ferritin variant polypeptide has a sequence according to SEQ ID NO. 82, SEQ ID NO. 1 or SEQ ID NO. 2, wherein at least one, preferably all, lysine residues at position 54, 72, 87 and/or 144 are deleted or substituted with a non-basic amino acid, preferably E or Q, and wherein the sequences according to SEQ ID NO. 82, SEQ ID NO. 1 and SEQ ID NO. 2 may further comprise 1-5, 1-10, 1-15, 1-20 or 1-25 amino acid mutations outside position 54, 72, 87 and/or 144.

10. The ferritin variant polypeptide of any one of claims 1 to 9, wherein one or more cysteine residues, in particular cysteine residues at position 91, 103 and/or 131 indicated with respect to SEQ ID NO. 1, are deleted or substituted, preferably substituted with serine residues.

11. The ferritin variant polypeptide of any one of claims 1 to 10, wherein the ferritin variant polypeptide has a sequence according to SEQ ID NO. 83, SEQ ID NO. 84, SEQ ID NO. 85, SEQ ID NO. 86, SEQ ID NO. 75, SEQ ID NO. 76, or SEQ ID NO. 77 or a sequence according to SEQ ID NO. 83, SEQ ID NO. 84, SEQ ID NO. 85, SEQ ID NO. 86, SEQ ID NO. 75, SEQ ID NO. 76, or SEQ ID NO. 77 comprising 1-5, 1-10, 1-15, 1-20 or 1-25 amino acid mutations outside position 54, 72, 87 and/or 144.

12. The ferritin variant polypeptide of any one of claims 1 to 11, further comprising a transferrin receptor binding domain (TRBD) of a ferritin variant wherein the TRBD in comparison to the wild-type ferritin on which it is based comprises one or more glutamine residues mutated into glutamic acid residues and/or one or more asparagine residues mutated into aspartic acid residues, wherein in particular at least one, preferably all mutations are comprised in the 20 N-terminal amino acids of the wild-type ferritin.

13. The ferritin variant polypeptide of claim 12, wherein the TRBD comprises at least the following amino acid sequence:
(SEQ ID NO. 3)
MTTASX₁SZVRZBYHZDX₂EAA
X₁=S or T, preferably T;
X₂=S or A, preferably S;
Z=Q or E; and
B=N or D;
wherein at least one Z or B is E or D,
which may further comprise one, two or three amino acid substitutions outside Z and/or B, and wherein the M at position 1 may be present or absent.

14. The ferritin variant polypeptide according to claim 12 or 13, wherein the TRBD comprises at least an amino acid sequence selected from the group comprising SEQ ID NO. 04 to SEQ ID NO. 63, particularly from the group consisting of SEQ ID NO. 05, 11, 12, 15, 20, 26, 27, 30, 35, 41, 42, 45, 50, 56, 57 and 60, more particularly from the group consisting of SEQ ID NO. 05, 12, 20, 27, 35, 42, 50 and 57, which may further comprise one, two or three amino acid substitutions outside amino acid positions 8, 11, 12 and/or 15, and wherein the M at position 1 may be present or absent.

15. The ferritin variant polypeptide according to any of claims 12 to 14, wherein the affinity of the TRBD to TfR-1 is increased in comparison to the TRBD of the wild-type ferritin at least (≥) 1.5×, ≥2×, ≥3×, ≥4×, ≥5×, ≥10×, ≥20×, ≥30×, ≥40×, ≥50×, but less than (≤) 100×, ≤75×≤50×, ≤40×, ≤30×, ≤20×, ≤10×, or ≤5×, in particular the affinity of the TRBD to TfR-1 is increased between 1.5×-50×, 2×-50×, 3×-50×, 4×-50×, 5×-50×, 10×-50×, 20×-50×, 30×-50×, 40×-50×, 1.5×-10×, 2×-20× or 5×-30× in comparison to the TRBD of the wild-type ferritin.

16. A nucleic acid encoding the polypeptide of any of claims 1 to 15.

17. A vector comprising the nucleic acid of claim 16.

18. A conjugate comprising the polypeptide of claims 1 to 15 and at least one label and/or at least one drug.

19. A complex comprising at least one polypeptide of claims 1 to 15 and/or at least one conjugate of claim 18.

20. The complex of claim 16 further comprising at least one label and/or at least one drug.

21. The conjugate of claim 18 or the complex of claim 19 or 20, wherein the label is selected from the group consisting of
a. a fluorescent dye, in particular a fluorescent dye selected from the group consisting of the following classes of fluorescent dyes: Xanthens, Acridines, Oxazines, Cynines, Styryl dyes, Coumarines, Porphines, Metal-Ligand-Complexes, Fluorescent proteins, Nanocrystals, Perylenes and Phtalocyanines as well as conjugates and combinations of these classes of dyes;
b. a radioisotope/fluorescence emitting isotope, in particular a radioisotope/fluorescence emitting isotope selected from the group consisting of alpha radiation emitting isotopes, gamma radiation emitting isotopes, Auger electron emitting isotopes, X-ray emitting isotopes, fluorescent isotopes, such as 65Tb, fluorescence emitting isotopes, such as 18F, 51Cr, 67Ga, 68Ga, 89Zr, 111In, 99mTc, 140La, 175Yb, 153Sm, 166Ho, 88Y, 90Y, 149Pm, 177Lu, 47Sc, 142Pr, 159Gd, 212Bi, 72As, 72Se, 97Ru, 109Pd, 105Rh, 101m15Rh, 119Sb, 128Ba, 123I, 124I, 131I, 197Hg, 211At, 169Eu, 203Pb, 212Pb, 64Cu, 67Cu, 188Re, 186Re, 198Au and 199Ag as well as conjugates and combinations of above with proteins, peptides, small molecular inhibitors, antibodies or other compounds;
c. a detectable polypeptide, in particular an autofluorescent protein, preferably green fluorescent protein or any structural variant thereof with an altered adsorption and/or emission spectrum or nucleic acid encoding a detectable polypeptide; and
d. a contrast agent, in particular a contrast agent comprising a paramagnetic agent, preferably selected from Gd, Eu, W and Mn, or ferrihydride.

22. The conjugate of claim 18 or 21 or the complex of claims 19 to 21, wherein the drug is selected from the group consisting of an anticancer drug, in particular a cytostatic drug, cytotoxic drug or prodrug thereof, an anti-arteriosclerotic drug, and an anti-inflammatory or immunomodulatory drug.

23. The conjugate of claim 18, 21 or 22 comprising a drug, wherein the drug is auristatin, in particular monomethyl auristatin (MMAE), conjugated to the polypeptide via a maleimidocaproyl-valine-citrulline-p-aminobenzoyloxycarbonyl linker.

24. An isolated targeted delivery system comprising a cell, wherein the cell comprises the polypeptide of claims 1 to 15, the conjugate of claim 18 or 21 to 23, or the complex of claims 19 to 23, wherein particularly the cell is a CD45+ leukocyte, more particularly a CD45+ leukocyte selected from the group consisting of a monocyte, a differentiated monocyte, lymphocyte and a granulocyte.

25. A pharmaceutical or diagnostic composition comprising the polypeptide of claims 1 to 15, the conjugate of claim 18 or 21 to 23 or the complex of claims 19 to 23 or the isolated targeted delivery system of claim 24 and a pharmaceutically acceptable carrier and/or suitable excipient(s).

26. The polypeptide of claims 1 to 15, the conjugate of claim 18 or 21 to 23 or the complex of claims 19 to 23 or the isolated targeted delivery system of claim 24 for use in medicine.


SEQUENCE LISTING (87 sequences)

SEQ ID NO: 1 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - protein, 182 aa, Mus musculus
MTTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDES

SEQ ID NO: 3 - protein, 20 aa, Artificial Sequence
  human/mouse
  misc_feature (6)..(6): S or T, preferably T
  misc_feature (8)..(8): Q or E
  misc_feature (11)..(11): Q or E
  misc_feature (12)..(12): N or D
  misc_feature (15)..(15): Q or E
  misc_feature (17)..(17): S or A, preferably S
MTTASXSZVRZBYHZDXEAA

SEQ ID NO: 4 - protein, 20 aa, Homo sapiens
MTTASTSEVRQNYHQDSEAA

SEQ ID NO: 5 - protein, 20 aa, Homo sapiens
MTTASTSQVRENYHQDSEAA

SEQ ID NO: 6 - protein, 20 aa, Homo sapiens
MTTASTSQVRQDYHQDSEAA

SEQ ID NO: 7 - protein, 20 aa, Homo sapiens
MTTASTSQVRQNYHEDSEAA

SEQ ID NO: 8 - protein, 20 aa, Homo sapiens
MTTASTSEVRENYHQDSEAA

SEQ ID NO: 9 - protein, 20 aa, Homo sapiens
MTTASTSEVRQDYHQDSEAA

SEQ ID NO: 10 - protein, 20 aa, Homo sapiens
MTTASTSEVRQNYHEDSEAA

SEQ ID NO: 11 - protein, 20 aa, Homo sapiens
MTTASTSQVREDYHQDSEAA

SEQ ID NO: 12 - protein, 20 aa, Homo sapiens
MTTASTSQVRENYHEDSEAA

SEQ ID NO: 13 - protein, 20 aa, Homo sapiens
MTTASTSQVRQDYHEDSEAA

SEQ ID NO: 14 - protein, 20 aa, Homo sapiens
MTTASTSEVREDYHQDSEAA

SEQ ID NO: 15 - protein, 20 aa, Homo sapiens
MTTASTSEVRENYHEDSEAA

SEQ ID NO: 16 - protein, 20 aa, Homo sapiens
MTTASTSEVRQDYHEDSEAA

SEQ ID NO: 17 - protein, 20 aa, Homo sapiens
MTTASTSQVREDYHEDSEAA

SEQ ID NO: 18 - protein, 20 aa, Homo sapiens
MTTASTSQVRQNYHQDSEAA

SEQ ID NO: 19 - protein, 20 aa, Mus musculus
MTTASSSEVRQNYHQDAEAA

SEQ ID NO: 20 - protein, 20 aa, Mus musculus
MTTASSSQVRENYHQDAEAA

SEQ ID NO: 21 - protein, 20 aa, Mus musculus
MTTASSSQVRQDYHQDAEAA

SEQ ID NO: 22 - protein, 20 aa, Mus musculus
MTTASSSQVRQNYHEDAEAA

SEQ ID NO: 23 - protein, 20 aa, Mus musculus
MTTASSSEVRENYHQDAEAA

SEQ ID NO: 24 - protein, 20 aa, Mus musculus
MTTASSSEVRQDYHQDAEAA

SEQ ID NO: 25 - protein, 20 aa, Mus musculus
MTTASSSEVRQNYHEDAEAA

SEQ ID NO: 26 - protein, 20 aa, Mus musculus
MTTASSSQVREDYHQDAEAA

SEQ ID NO: 27 - protein, 20 aa, Mus musculus
MTTASSSQVRENYHEDAEAA

SEQ ID NO: 28 - protein, 20 aa, Mus musculus
MTTASSSQVRQDYHEDAEAA

SEQ ID NO: 29 - protein, 20 aa, Mus musculus
MTTASSSEVREDYHQDAEAA

SEQ ID NO: 30 - protein, 20 aa, Mus musculus
MTTASSSEVRENYHEDAEAA

SEQ ID NO: 31 - protein, 20 aa, Mus musculus
MTTASSSEVRQDYHEDAEAA

SEQ ID NO: 32 - protein, 20 aa, Mus musculus
MTTASSSQVREDYHEDAEAA

SEQ ID NO: 33 - protein, 20 aa, Mus musculus
MTTASSSQVRQNYHQDAEAA

SEQ ID NO: 34 - protein, 20 aa, Artificial Sequence
  human/mouse
MTTASTSEVRQNYHQDAEAA

SEQ ID NO: 35 - protein, 20 aa, Artificial Sequence
  human/mouse
MTTASTSQVRENYHQDAEAA

SEQ ID NO: 36 - protein, 20 aa, Artificial Sequence
  human/mouse
MTTASTSQVRQDYHQDAEAA

SEQ ID NO: 37 - protein, 20 aa, Artificial Sequence
  human/mouse
MTTASTSQVRQNYHEDAEAA

SEQ ID NO: 38 - protein, 20 aa, Artificial Sequence
  human/mouse
MTTASTSEVRENYHQDAEAA

SEQ ID NO: 39 - protein, 20 aa, Artificial Sequence
  human/mouse
MTTASTSEVRQDYHQDAEAA

SEQ ID NO: 40 - protein, 20 aa, Artificial Sequence
  human/mouse
MTTASTSEVRQNYHEDAEAA

SEQ ID NO: 41 - protein, 20 aa, Artificial Sequence
  human/mouse
MTTASTSQVREDYHQDAEAA

SEQ ID NO: 42 - protein, 20 aa, Artificial Sequence
  human/mouse
MTTASTSQVRENYHEDAEAA

SEQ ID NO: 43 - protein, 20 aa, Artificial Sequence
  human/mouse
MTTASTSQVRQDYHEDAEAA

SEQ ID NO: 44 - protein, 20 aa, Artificial Sequence
  human/mouse
MTTASTSEVREDYHQDAEAA

SEQ ID NO: 45 - protein, 20 aa, Artificial Sequence
  human/mouse
MTTASTSEVRENYHEDAEAA

SEQ ID NO: 46 - protein, 20 aa, Artificial Sequence
  human/mouse
MTTASTSEVRQDYHEDAEAA

SEQ ID NO: 47 - protein, 20 aa, Artificial Sequence
  human/mouse
MTTASTSQVREDYHEDAEAA

SEQ ID NO: 48 - protein, 20 aa, Artificial Sequence
  human/mouse
MTTASTSQVRQNYHQDAEAA

SEQ ID NO: 49 - protein, 20 aa, Artificial Sequence
  human/mouse
MTTASSSEVRQNYHQDSEAA

SEQ ID NO: 50 - protein, 20 aa, Artificial Sequence
  human/mouse
MTTASSSQVRENYHQDSEAA

SEQ ID NO: 51 - protein, 20 aa, Artificial Sequence
  human/mouse
MTTASSSQVRQDYHQDSEAA

SEQ ID NO: 52 - protein, 20 aa, Artificial Sequence
  human/mouse
MTTASSSQVRQNYHEDSEAA

SEQ ID NO: 53 - protein, 20 aa, Artificial Sequence
  human/mouse
MTTASSSEVRENYHQDSEAA

SEQ ID NO: 54 - protein, 20 aa, Artificial Sequence
  human/mouse
MTTASSSEVRQDYHQDSEAA

SEQ ID NO: 55 - protein, 20 aa, Artificial Sequence
  human/mouse
MTTASSSEVRQNYHEDSEAA

SEQ ID NO: 56 - protein, 20 aa, Artificial Sequence
  human/mouse
MTTASSSQVREDYHQDSEAA

SEQ ID NO: 57 - protein, 20 aa, Artificial Sequence
  human/mouse
MTTASSSQVRENYHEDSEAA

SEQ ID NO: 58 - protein, 20 aa, Artificial Sequence
  human/mouse
MTTASSSQVRQDYHEDSEAA

SEQ ID NO: 59 - protein, 20 aa, Artificial Sequence
  human/mouse
MTTASSSEVREDYHQDSEAA

SEQ ID NO: 60 - protein, 20 aa, Artificial Sequence
  human/mouse
MTTASSSEVRENYHEDSEAA

SEQ ID NO: 61 - protein, 20 aa, Artificial Sequence
  human/mouse
MTTASSSEVRQDYHEDSEAA

SEQ ID NO: 62 - protein, 20 aa, Artificial Sequence
  human/mouse
MTTASSSQVREDYHEDSEAA

SEQ ID NO: 63 - protein, 20 aa, Artificial Sequence
  human/mouse
MTTASSSQVRQNYHQDSEAA

SEQ ID NO: 64 - protein, 163 aa, Artificial Sequence
  mammalian consensus
  misc_feature (1)..(2): Xaa can be any naturally occurring amino acid
  misc_feature (10)..(10): Xaa can be any naturally occurring amino acid
  misc_feature (20)..(20): Xaa can be any naturally occurring amino acid
  misc_feature (62)..(62): Xaa can be any naturally occurring amino acid
  misc_feature (64)..(64): Xaa can be any naturally occurring amino acid
  misc_feature (71)..(71): Xaa can be any naturally occurring amino acid
  misc_feature (86)..(86): Xaa can be any naturally occurring amino acid
  misc_feature (90)..(90): Xaa can be any naturally occurring amino acid
  misc_feature (117)..(117): Xaa can be any naturally occurring amino acid
  misc_feature (120)..(120): Xaa can be any naturally occurring amino acid
  misc_feature (125)..(125): Xaa can be any naturally occurring amino acid
  misc_feature (144)..(144): Xaa can be any naturally occurring amino acid
  misc_feature (146)..(146): Xaa can be any naturally occurring amino acid
  misc_feature (158)..(158): Xaa can be any naturally occurring amino acid
  misc_feature (161)..(163): Xaa can be any naturally occurring amino acid
XXRQINLELXASYVYLSMSXYFDRDDVALKNFAKYFLHQSHEEREHAEKLMKLQNQRGGRIXLXDIKKPDXDDWESGLNAMECALXLEKXVNQSLLELHKLATDKNDPHLCDFIETXYLXEQVKXIKELGDHVTNLRKMGAPEXGXAEYLFDKHTLGXSDXXX

SEQ ID NO: 65 - protein, 129 aa, Artificial Sequence
  mammalian consensus
  misc_feature (1)..(2): Xaa can be any naturally occurring amino acid
  MISC_FEATURE (6)..(6): any naturally occurring amino acid, preferably Pro
  misc_feature (10)..(10): Xaa can be any naturally occurring amino acid
  MISC_FEATURE (14)..(14): any naturally occurring amino acid, preferably His
  MISC_FEATURE (16)..(16): any naturally occurring amino acid, preferably Asp
  misc_feature (20)..(20): Xaa can be any naturally occurring amino acid
  MISC_FEATURE (21)..(21): may be present or absent, if present it means any naturally occurring amino acid, preferably Ile
  MISC_FEATURE (22)..(22): any naturally occurring amino acid, preferably Asn
  MISC_FEATURE (30)..(30): any naturally occurring amino acid, preferably Tyr
  MISC_FEATURE (40)..(40): any naturally occurring amino acid, preferably Tyr or Cys, more preferably Cys
  misc_feature (62)..(62): Xaa can be any naturally occurring amino acid
  misc_feature (64)..(64): Xaa can be any naturally occurring amino acid
  misc_feature (71)..(71): Xaa can be any naturally occurring amino acid
  MISC_FEATURE (82)..(82): any naturally occurring amino acid, preferably Phe
  MISC_FEATURE (84)..(84): any naturally occurring amino acid, preferably Gln
  misc_feature (86)..(86): Xaa can be any naturally occurring amino acid
  misc_feature (90)..(90): Xaa can be any naturally occurring amino acid
  MISC_FEATURE (91)..(91): any naturally occurring amino acid, preferably Arg or Cys, more preferably Cys
  MISC_FEATURE (106)..(106): any naturally occurring amino acid, preferably His
  MISC_FEATURE (110)..(110): any naturally occurring amino acid, preferably Asn or Ser, more preferably Asn
  misc_feature (117)..(117): Xaa can be any naturally occurring amino acid
  misc_feature (120)..(120): Xaa can be any naturally occurring amino acid
  misc_feature (125)..(125): Xaa can be any naturally occurring amino acid
  MISC_FEATURE (137)..(137): any naturally occurring amino acid, preferably His or Tyr, more preferably His
  MISC_FEATURE (140)..(140): any naturally occurring amino acid, preferably Asn or Ser, more preferably Asn
  MISC_FEATURE (145)..(145): any naturally occurring amino acid, preferably Ala or Ser, more preferably Ala
  MISC_FEATURE (164)..(164): any naturally occurring amino acid, preferably Ala or Ser, more preferably Ser
  MISC_FEATURE (166)..(166): any naturally occurring amino acid, preferably Met or Leu, more preferably Leu
  MISC_FEATURE (178)..(178): any naturally occurring amino acid, preferably Asp or His, more preferably Asp
  MISC_FEATURE (181)..(181): may be present or absent, if present it means any amino acid, preferably Asn
  MISC_FEATURE (182)..(182): may be present or absent, if present it means any amino acid, preferably Glu
  MISC_FEATURE (183)..(183): may be present or absent, if present it means any amino acid, preferably Ser
XXRQINLELXASYVYLSMSXYFDRDDVALKNFAKYFLHQSHEEREHAEKLMKLQNQRGGRIXLXDIKKPDXDDWESGLNAMECALXLEKXVNQSLLELHKLATDKNDPHLCDFIETXYLXEQVKXIKEL

SEQ ID NO: 66 - protein, 162 aa, Artificial Sequence
  mammalian consensus
  misc_feature (1)..(1): Xaa can be any naturally occurring amino acid
  misc_feature (9)..(9): Xaa can be any naturally occurring amino acid
  misc_feature (61)..(61): Xaa can be any naturally occurring amino acid
  misc_feature (63)..(63): Xaa can be any naturally occurring amino acid
  misc_feature (85)..(85): Xaa can be any naturally occurring amino acid
  misc_feature (124)..(124): Xaa can be any naturally occurring amino acid
  misc_feature (160)..(162): Xaa can be any naturally occurring amino acid
XRQINLELXASYVYLSMSYYFDRDDVALKNFAKYFLHQSHEEREHAEKLMKLQNQRGGRIXLXDIKKPDCDDWESGLNAMECALXLEKNVNQSLLELHKLATDKNDPHLCDFIETHYLNEQVKXIKELGDHVTNLRKMGAPESGLAEYLFDKHTLGDSDXXX

SEQ ID NO: 67 - protein, 163 aa, Homo sapiens
INRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 68 - protein, 162 aa, Mus musculus
INRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDES

SEQ ID NO: 69 - protein, 162 aa, Rattus norvegicus
INRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETHYLNEQVKSIKELGDHVTNLRKMGAPESGMAEYLFDKHTLGHGDES

SEQ ID NO: 70 - protein, 163 aa, Oryctolagus cuniculus
INRQINLELYASYVYLSMSYYFDRDDVALKNFAKYFLHQSHEEREHAEKLMKLQNQRGGRIFLQDIKKPEYDDWESGLNAMECALHLEKSVNQSLLELHKLATDKNDPHLCDFIETHYLNEQVKSIKELGDHVTNLRKMGAPESGMAEYLFDKHTLGHSDNES

SEQ ID NO: 71 - protein, 183 aa, Homo sapiens
MTTASTSQVRENYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 72 - protein, 183 aa, Homo sapiens
MTTASTSQVRENYHEDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 73 - protein, 183 aa, Homo sapiens
MTTASTSEVRENYHEDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 74 - protein, 183 aa, Homo sapiens
MTTASTSQVREDYHQDSEAAINRQINLELYASYVYLSMSYYFDRDDVALKNFAEYFLHQSHEEREHAEKLMELQNQRGGRIFLQDIQKPDSDDWESGLNAMESALHLEKNVNQSLLELHKLATDKNDPHLSDFIETHYLNEQVEAIKELGDHVTNLRKMGAPESGLAEYLFDKHTLGDSDNES

SEQ ID NO: 75 - protein, 183 aa, Homo sapiens
MTTASTSQVRENYHQDSEAAINRQINLELYASYVYLSMSYYFDRDDVALKNFAEYFLHQSHEEREHAEKLMELQNQRGGRIFLQDIQKPDSDDWESGLNAMESALHLEKNVNQSLLELHKLATDKNDPHLSDFIETHYLNEQVEAIKELGDHVTNLRKMGAPESGLAEYLFDKHTLGDSDNES

SEQ ID NO: 76 - protein, 183 aa, Homo sapiens
MTTASTSQVRENYHEDSEAAINRQINLELYASYVYLSMSYYFDRDDVALKNFAEYFLHQSHEEREHAEKLMELQNQRGGRIFLQDIQKPDSDDWESGLNAMESALHLEKNVNQSLLELHKLATDKNDPHLCDFIETHYLNEQVEAIKELGDHVTNLRKMGAPESGLAEYLFDKHTLGDSDNES

SEQ ID NO: 77 - protein, 183 aa, Homo sapiens
MTTASTSQVRENYHEDSEAAINRQINLELYASYVYLSMSYYFDRDDVALKNFAEYFLHQSHEEREHAEKLMELQNQRGGRIFLQDIQKPDCDDWESGLNAMECALHLEKNVNQSLLELHKLATDKNDPHLCDFIETHYLNEQVEAIKELGDHVTNLRKMGAPESGLAEYLFDKHTLGDSDNES

SEQ ID NO: 78 - protein, 163 aa, Homo sapiens
INRQINLELYASYVYLSMSYYFDRDDVALKNFAEYFLHQSHEEREHAEKLMELQNQRGGRIFLQDIQKPDSDDWESGLNAMESALHLEKNVNQSLLELHKLATDKNDPHLSDFIETHYLNEQVEAIKELGDHVTNLRKMGAPESGLAEYLFDKHTLGDSDNES

SEQ ID NO: 79 - protein, 163 aa, Homo sapiens
INRQINLELYASYVYLSMSYYFDRDDVALKNFAEYFLHQSHEEREHAEKLMELQNQRGGRIFLQDIQKPDSDDWESGLNAMESALHLEKNVNQSLLELHKLATDKNDPHLCDFIETHYLNEQVEAIKELGDHVTNLRKMGAPESGLAEYLFDKHTLGDSDNES

SEQ ID NO: 80 - protein, 163 aa, Homo sapiens
INRQINLELYASYVYLSMSYYFDRDDVALKNFAEYFLHQSHEEREHAEKLMELQNQRGGRIFLQDIQKPDCDDWESGLNAMECALHLEKNVNQSLLELHKLATDKNDPHLCDFIETHYLNEQVEAIKELGDHVTNLRKMGAPESGLAEYLFDKHTLGDSDNES

SEQ ID NO: 81 - protein, 20 aa, unknown
  human mouse consensus
  MISC_FEATURE (6)..(6): X1 S or T, preferably T
  MISC_FEATURE (8)..(8): Z1 Q or E
  MISC_FEATURE (11)..(11): Z2 Q or E
  MISC_FEATURE (12)..(12): B N or D
  MISC_FEATURE (15)..(15): Z3 Q or E
  MISC_FEATURE (17)..(17): X2 S or A, preferably S
MTTASXSZVRZBYHZDXEAA

SEQ ID NO: 82 - protein, 183 aa, unknown
  human mouse consensus
  misc_feature (6)..(6): Xaa can be any naturally occurring amino acid
  misc_feature (14)..(14): Xaa can be any naturally occurring amino acid
  misc_feature (16)..(16): Xaa can be any naturally occurring amino acid
  misc_feature (21)..(22): Xaa can be any naturally occurring amino acid
  misc_feature (30)..(30): Xaa can be any naturally occurring amino acid
  misc_feature (40)..(40): Xaa can be any naturally occurring amino acid
  misc_feature (82)..(82): Xaa can be any naturally occurring amino acid
  misc_feature (84)..(84): Xaa can be any naturally occurring amino acid
  misc_feature (91)..(91): Xaa can be any naturally occurring amino acid
  misc_feature (106)..(106): Xaa can be any naturally occurring amino acid
  misc_feature (110)..(110): Xaa can be any naturally occurring amino acid
  misc_feature (137)..(137): Xaa can be any naturally occurring amino acid
  misc_feature (140)..(140): Xaa can be any naturally occurring amino acid
  misc_feature (145)..(145): Xaa can be any naturally occurring amino acid
  misc_feature (164)..(164): Xaa can be any naturally occurring amino acid
  misc_feature (166)..(166): Xaa can be any naturally occurring amino acid
  misc_feature (178)..(178): Xaa can be any naturally occurring amino acid
  misc_feature (181)..(183): Xaa can be any naturally occurring amino acid
MTTASXSQVRQNYXQXSEAAXXRQINLELXASYVYLSMSXYFDRDDVALKNFAKYFLHQSHEEREHAEKLMKLQNQRGGRIXLXDIKKPDXDDWESGLNAMECALXLEKXVNQSLLELHKLATDKNDPHLCDFIETXYLXEQVKXIKELGDHVTNLRKMGAPEXGXAEYLFDKHTLGXSDXXX

SEQ ID NO: 83 - protein, 183 aa, Homo sapiens
MTTASTSQVRQNYHQDSEAAINRQINLELYASYVYLSMSYYFDRDDVALKNFAEYFLHQSHEEREHAEKLMELQNQRGGRIFLQDIQKPDCDDWESGLNAMECALHLEKNVNQSLLELHKLATDKNDPHLCDFIETHYLNEQVEAIKELGDHVTNLRKMGAPESGLAEYLFDKHTLGDSDNES

SEQ ID NO: 84 - protein, 183 aa, Homo sapiens
MTTASTSQVRQNYHQDSEAAINRQINLELYASYVYLSMSYYFDRDDVALKNFAZYFLHQSHEEREHAEKLMZLQNQRGGRIFLQDIZKPDCDDWESGLNAMECALHLEKNVNQSLLELHKLATDKNDPHLCDFIETHYLNEQVZAIKELGDHVTNLRKMGAPESGLAEYLFDKHTLGDSDNES

SEQ ID NO: 85 - protein, 183 aa, Homo sapiens
MTTASTSQVRENYHEDSEAAINRQINLELYASYVYLSMSYYFDRDDVALKNFAZYFLHQSHEEREHAEKLMZLQNQRGGRIFLQDIZKPDCDDWESGLNAMECALHLEKNVNQSLLELHKLATDKNDPHLCDFIETHYLNEQVZAIKELGDHVTNLRKMGAPESGLAEYLFDKHTLGDSDNES

SEQ ID NO: 86 - protein, 183 aa, Homo sapiens
  misc_feature (6)..(6): Xaa can be any naturally occurring amino acid
  misc_feature (14)..(14): Xaa can be any naturally occurring amino acid
  misc_feature (16)..(16): Xaa can be any naturally occurring amino acid
  misc_feature (21)..(22): Xaa can be any naturally occurring amino acid
  misc_feature (30)..(30): Xaa can be any naturally occurring amino acid
  misc_feature (40)..(40): Xaa can be any naturally occurring amino acid
  misc_feature (82)..(82): Xaa can be any naturally occurring amino acid
  misc_feature (84)..(84): Xaa can be any naturally occurring amino acid
  misc_feature (91)..(91): Xaa can be any naturally occurring amino acid
  misc_feature (106)..(106): Xaa can be any naturally occurring amino acid
  misc_feature (110)..(110): Xaa can be any naturally occurring amino acid
  misc_feature (137)..(137): Xaa can be any naturally occurring amino acid
  misc_feature (140)..(140): Xaa can be any naturally occurring amino acid
  misc_feature (145)..(145): Xaa can be any naturally occurring amino acid
  misc_feature (164)..(164): Xaa can be any naturally occurring amino acid
  misc_feature (166)..(166): Xaa can be any naturally occurring amino acid
  misc_feature (178)..(178): Xaa can be any naturally occurring amino acid
  misc_feature (181)..(183): Xaa can be any naturally occurring amino acid
MTTASXSQVRQNYXQXSEAAXXRQINLELXASYVYLSMSXYFDRDDVALKNFAZYFLHQSHEEREHAEKLMZLQNQRGGRIXLXDIZKPDXDDWESGLNAMECALXLEKXVNQSLLELHKLATDKNDPHLCDFIETXYLXEQVZXIKELGDHVTNLRKMGAPEXGXAEYLFDKHTLGXSDXXX

SEQ ID NO: 87 - protein, 163 aa, Homo sapiens
INRQINLELYASYVYLSMSYYFDRDDVALKNFAZYFLHQSHEEREHAEKLMZLQNQRGGRIFLQDIZKPDCDDWESGLNAMECALHLEKNVNQSLLELHKLATDKNDPHLCDFIETHYLNEQVZAIKELGDHVTNLRKMGAPESGLAEYLFDKHTLGDSDNES